(19) United States
(12) Patent Application Publication
(10) Pub. No.: US 2019/0122759 A1
(43) Pub. Date: Apr. 25, 2019

(54) IN-FACILITY MONITORING SYSTEM, IN-FACILITY MONITORING APPARATUS, AND COMPUTER PROGRAM

(71) Applicant: SYSMEX CORPORATION, Kobe-shi (JP)

(72) Inventor: Tatsuru Wakimoto, Itoshima-shi (JP)

(73) Assignee: SYSMEX CORPORATION, Kobe-shi (JP)

(21) Appl. No.: 16/160,232

(22) Filed: Oct. 15, 2018

(30) Foreign Application Priority Data
Oct. 20, 2017 (JP) 2017-203934

(51) Int. Cl. G16H 40/20; G16H 50/80; G01C 21/20

(52) U.S. Cl. CPC G16H 40/20; G01C 21/206; G16H 50/80

(57) ABSTRACT

A system is provided for comprehending the risk of occurrence of disease arising from environmental conditions by monitoring the environmental conditions in a facility. The present disclosure describes an in-facility monitoring system including a display unit for displaying an area layout diagram showing the positions of a plurality of areas in the facility, sensors provided in each of the areas to measure environmental parameters related to the risk of the occurrence of disease, and a control unit for generating display data for identifiably displaying the environmental conditions representing the risk of occurrence of disease in each area in the area layout diagram based on the measured values of the environmental parameter output from the sensors.

### CROSS REFERENCE TO RELATED APPLICATIONS

This application claims priority from prior Japanese Patent Application Publication No. 2017-203934, filed on Oct. 20, 2017, entitled IN-FACILITY MONITORING SYSTEM, IN-FACILITY MONITORING APPARATUS, AND COMPUTER PROGRAM ”, the entire contents of which are incorporated herein by reference.

### FIELD OF THE INVENTION

The present invention relates to an in-facility monitoring system, an in-facility monitoring apparatus, and a computer program.

### BACKGROUND

Japanese Patent Application Publication No. 2012-226571 describes a system for monitoring nosocomial infection (FIG. 19A). According to the infection information provision system described in Japanese Patent Application Publication No. 2012-226571, pathogen identification information for identifying a pathogen infecting a patient can be identified in a ward map (FIG. 19B) or the like indicating the placement position of the ward in the hospital facility.

### SUMMARY OF THE INVENTION

It is known that various diseases occur due to deterioration of environmental conditions such as temperature and humidity inside facilities. That is, for example, when temperature and humidity are particularly involved in influenza outbreaks prevalent from December to March, keeping the inside of the facility at moderate humidity (50 to 60%) is said to effectively prevent influenza virus infection. On the other hand, heat stroke may occur in a room where a summer air conditioner is not used, and mortality among the elderly has been reported in severe cases.

Monitoring the environmental conditions in the facility and maintaining appropriate environmental conditions is important in preventing the spread of infectious diseases and ailments caused by environmental factors such as heat stroke. However, in the system described in Japanese Patent Application Publication No. 2012-226571, it is not possible to comprehend the danger of disease arising from environmental conditions within the facility since the information of in-hospital infections is displayed so as to be identifiable on the basis of the information of the pathogen already infecting the patient.

In view of the above prior art, the present invention can grasp the risk of the occurrence of diseases caused by environmental conditions by monitoring the environmental condition in the facility. Another aim is to provide a method of allowing a user to quickly comprehend environmental conditions in each section such as the rooms of the patients.

One aspect of the invention relates to an in-facility monitoring system (100) provided with a display device (32) for displaying an area layout diagram showing the positions of a plurality of areas in a facility, sensors (60) provided in each area to measure environment parameters (50) associated with the risk of occurrence of disease, a control unit (21a) for generating display data (40a) for identifiably displaying environmental conditions representing the risk of occurrence of disease in each area in the area layout diagram based on the measured value of the environmental parameter (50) output from the sensor. According to this aspect, it is possible to monitor the environmental conditions in the facility by the sensors and generate display data for allowing the user to comprehend the risk of the occurrence of a disease arising from the environmental condition.

In one aspect of the present invention, the in-facility monitoring system (100) further includes a storage unit; the storage unit (21d) stores a reference range of the environmental parameter, and the control unit (21a) determines that an environmental condition is sub-nominal for each area in which the measured value of the environmental parameter (50) is outside the reference range. According to this aspect, it is possible to generate display data for comprehending the area in which the environmental condition is sub-nominal.

In one aspect of the present invention, the in-facility monitoring system (100) further includes a storage unit, the storage unit (21d) stores a reference range of the environmental parameter, and the control unit (21a) requests an index indicating the risk of occurrence of disease in each area from the measured value of parameter (50), and determines that the environmental condition is sub-nominal for an area in which the index exceeds the reference range. According to this aspect, it is possible to generate display data for comprehending an area in which the environmental condition is sub-nominal from the index by obtaining an index indicating the environmental condition from a plurality of environmental parameters (50).

In one aspect of the invention, the storage unit (21d) stores a reference range corresponding to the type of disease. According to this aspect, it is possible to generate display data for comprehending the environmental condition corresponding to the disease by defining a reference range for each disease.

In the in-facility monitoring system (100) of one aspect of the invention, the environmental parameter (50) is at least one selected from the group including temperature information, humidity information, and ventilation information. According to this aspect, it is possible to generate display data for comprehending the environmental condition particularly for diseases related to temperature, humidity, or ventilation frequency.

In the in-facility monitoring system (100) of one aspect of the invention, the disease is an infectious disease or a heat stroke. According to this aspect, it is possible to generate display data for specifically comprehending an environmental condition related to infectious diseases or heat stroke.

In the invention is the in-facility monitoring system (100) of one aspect of the invention, the infection is a droplet infection. According to this aspect, it is possible to generate display data especially for comprehending the environmental conditions related to droplet infection.

One aspect of the invention is the in-facility monitoring system (100), wherein the droplet infectious disease is caused by at least one selected from the group including viruses, fungi, and bacteria. According to this aspect, it is possible to generate display data for specifically comprehending environmental conditions related to viruses, fungi, or bacteria.

In the in-facility monitoring system (100) of one aspect of the invention, the virus is at least one type selected from a group including influenza virus, RS virus, adenovirus, rhinovirus, coronavirus, and parainfluenz, the fungus is at least one type selected from a group including Candida, Actinomyces, Geotrichum, Aspergillus, Cryptococcus, and Nocardia, the bacterium is at least one type selected from a group including Mycoplasma, Legionella, Haemophilus, Pneumococcus, and Bordetella pertussis. According to this aspect, it is possible to generate display data for comprehending the environmental conditions related to the pathogens.

In the in-facility monitoring system (100) of one aspect of the invention, the sensors measure the environmental parameter (50) over time, and the control unit (21a) records the measured values of the environmental parameter (50) measured over time by the sensors in the storage unit, and determines whether the environmental condition of each area is nominal based on the measured value of the environmental parameter (50) over time. According to this aspect, it is possible to generate display data for comprehending the environmental conditions related to the occurrence of disease based on the result of monitoring the environmental conditions over time.

In the in-facility monitoring system (100) of one aspect of the invention, the control unit (21a) generates caution alert data to identifiably display a caution call level according to the environmental condition of each area in the in-facility area diagram. According to this aspect, it is possible to generate caution alert data to allow the user to comprehend the attention call level corresponding to the environmental condition of each are.

In the in-facility monitoring system (100) according to one aspect of the invention, the attention call level is identifiably displayed so as to be distinguishable by means of at least one item selected from among marks, colors, icons, characters, and numerical values. According to this aspect of the invention, the user can comprehend at a glance in which area the environmental condition may have become unsatisfactory on the area payout diagram.

In the in-facility monitoring system (100) according to one aspect of the invention, when there is an area with a sub-nominal environmental condition, the control unit (21a) notifies a preset mobile terminal (400) of the environmental condition of that area. According to this aspect, the user can comprehend that there is an area in which the environmental condition is sub-nominal without approaching the vicinity of the display unit (32), for example.

In the in-facility monitoring system (100) of one aspect of the invention, when there is an area in which the environmental condition is sub-nominal, the control unit (21a) displays the environmental parameter (50) of the corresponding area on the display unit. According to this aspect, the user can comprehend at a glance which environmental parameter must be controlled to become appropriate.

In an in-facility monitoring system (100) of one aspect of the invention, the facility can accommodate a plurality of people. In a facility capable of accommodating a plurality of people according to this aspect, it is possible to generate display data for allowing the user to comprehend the environmental conditions. This helps to prevent mass infection and the like.

In the in-facility monitoring system (100) of one aspect of the invention, the facility is at least one selected from a group including a medical facility, an educational facility, and a welfare facility. According to this aspect, it is possible to generate display data for the user to comprehend the environmental conditions in a facility where special attention should be paid to the spread of diseases, such as medical facilities, educational facilities, and welfare facilities. This helps to prevent mass infection and the like.

In the in-facility monitoring system (100) of one aspect of the invention, the facility is a medical facility and the area is a room. In a medical facility according to this aspect, it is possible to generate display data for allowing the user to comprehend the environmental conditions. This will help prevent the occurrence of new diseases in hospitals.

In the invention is the in-facility monitoring system (100) of one aspect of the invention, the infection is a nosocomial infection. In a medical facility according to this aspect, it is possible to generate display data for allowing the user to comprehend the environmental conditions. This helps to prevent nosocomial infection.

In the facility monitoring system (100) of one aspect of the invention, personal information related to a person staying in the area is displayed in the area layout diagram. According to this aspect, it is possible to simultaneously comprehend an area where the environmental conditions are sub-nominal and the kind of person staying in the area.

In the in-facility monitoring system (100) of one aspect of the invention, the personal information includes information concerning the disease afflicting the person. According to this aspect, it is possible to simultaneously comprehend an area where the environmental conditions are sub-nominal and the disease afflicting the person staying in the area.

An aspect of the invention is an in-facility monitoring apparatus (200) provided with a storage unit (21d) and a control unit (21a), wherein the apparatus (200) is connected to a client device (300), and the storage unit (21d) stores an area layout diagram showing the positions of a plurality of areas in the facility, and the control unit acquires an environmental parameter (50) related to the risk of the occurrence of a disease for each area output from a sensor (60) provided in each area, generates display data (40) for identifiably displaying environmental conditions representing the degree of risk of occurrence of disease in each area in the area layout diagram based on the acquired environmental parameter (50), and transmitting the generated display data to the client apparatus (300). According to this aspect, it is possible to monitor the environmental conditions in the facility by the sensors, and generate display data for allowing the user to comprehend the risk of occurrence of a disease arising from the environmental conditions.

An aspect of the invention relates to a program for causing a computer to function as an in-facility monitoring apparatus (200) provided with a control unit (21a) and a storage unit (21d) for storing an area layout diagram indicating the positions of a plurality of areas in the facility, the apparatus being connected to a client apparatus (300) that has a display unit, the program causes the control unit (21a) to execute a process for acquiring environmental parameters (50) associated with the degree of risk of occurrence of a disease output from a sensor (60) provided in each area, a process for generating display data (40) for identifiably displaying an environmental condition representing the degree of risk of occurrence of disease in each area in the area layout diagram based on the acquired environmental parameters (50) of each area, and a process of transmitting the generated display data to the client device (300) for display on the display unit (32). According to this aspect, it is possible to monitor the environmental conditions in the facility by the sensors, and generate display data for allowing the user to comprehend the risk of occurrence of a disease arising from the environmental conditions.

One aspect of the invention relates to an in-facility monitoring method including a step of generating display data for identifiably displaying environmental conditions indicating the risk of occurrence of disease in each area in an area layout diagram showing the position of each area based on the measured values of the environmental parameters (50) related to the risk of occurrence of disease in each area of the facility acquired from the sensors (60) provided for each of the plurality of areas in the facility, and a step of identifiably displaying the environmental conditions on the area layout diagram. According to this aspect, it is possible to generate display data for allowing the user to comprehend the risk of occurrence of diseases caused by the environmental conditions based on the data obtained by monitoring the environmental conditions in the facility via the sensors.

According to the present invention, it is possible to monitor the risk of occurrence of diseases caused by environmental conditions based on the environmental parameters of each area. The user also can promptly comprehend the environmental conditions of each area such as a patient's room.

### BRIEF DESCRIPTION OF THE DRAWINGS

FIG. 1 is an example of screen display in a client device of an in-facility monitoring system according to an embodiment;

FIG. 2 is a schematic diagram showing the overall configuration of the in-facility monitoring system according to the embodiment;

FIG. 3 is a block diagram showing a hardware configuration of an in-facility monitoring apparatus;

FIG. 4 is a schematic diagram showing a configuration of various databases stored in a storage unit of an in-facility monitoring apparatus;

FIG. 5 is a block diagram showing a hardware configuration of a client device;

FIG. 6 is a block diagram showing a hardware configuration of a portable information terminal;

FIG. 7 is an example of a screen display of a menu screen;

FIG. 8 is an example of a screen display of a ward map;

FIG. 9 is an example of a screen display of a floor map;

FIG. 10 is an example of a screen display of an area map;

FIG. 11 is a flowchart showing a procedure of a facility map display process;

FIG. 12 is an example of a screen display of a ward map;

FIG. 13 is an example of a screen display of a floor map;

FIG. 14 is an example of a screen display of an area map.

FIG. 15 is a flowchart showing a procedure of a caution alert display process;

FIG. 16A is a table showing a temperature reference range and a determination result; FIG. 16B is a table showing a humidity reference range and a determination result; FIG. 16C is a table showing a reference range of combined temperature and humidity, and a judgment result; FIG. 16D is a table showing measured values and determination results recorded over time;

FIG. 17 is a diagram showing an example of a heat index;

FIG. 18 is an example showing items of environmental parameters determined to be sub-nominal when a caution alert is covered with the pointer via a mouse;

FIG. 19 is a diagram showing a conventional technique described in Japanese Patent Application Publication No. 2012-226571; FIG. 19A indicates the overall configuration of the system; FIG. 19B is an example of a ward map; and

FIG. 20 is a display example of an input screen when a user optionally sets a reference range.

### DESCRIPTION OF THE EMBODIMENTS OF THE INVENTION

Hereinafter, embodiments of the invention will be described in detail with reference to the accompanying drawings. In the following description and drawings the same reference numerals denote the same or similar constituent elements, and therefore, the description of the same or similar constituent elements is omitted. Note that the invention is not limited to the specific embodiments described below.

### SUMMARY OF THE INVENTION

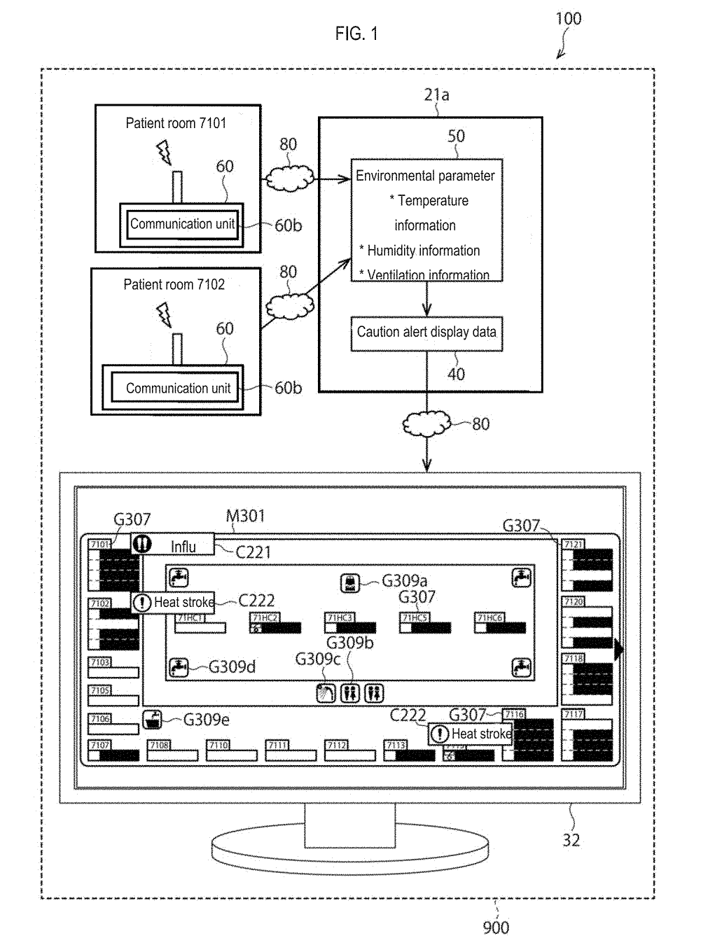

An outline of an embodiment of the invention will be described first with reference to FIG. 1. The in-facility monitoring system 100 (hereinafter simply referred to as the system 100) according to an embodiment of the invention is used for monitoring the condition of the environment in a facility 900. The environmental condition is, for example, an environmental condition related to the occurrence of a disease, and reflects the risk of occurrence of an environment-dependent disease. The environmental condition is preferably indicated by measurement information for example, measurement value) of an environmental factor (temperature, humidity, number of times of ventilation and light (for example, ultraviolet light and the like)). More preferably, the environmental condition indicates at least one condition selected from temperature, humidity, number of times of ventilation, light (for example, ultraviolet light) and the like. Still more preferably, the environmental condition indicates at least one condition selected from temperature and humidity. In this embodiment, the disease can be caused by deterioration of the environmental parameter 50, which is preferably related to the temperature, humidity, the number of times of ventilation, and the amount of light (for example ultraviolet light)and the like in the facility. Examples of such diseases include heat stroke or infectious diseases. Infectious diseases can be infectious diseases which are likely to lead to outbreaks such as droplet infections. Pathogens which readily produce droplet infection include viruses such as influenza virus, RS virus, adenovirus, rhinovirus, coronavirus, and parainfluenza virus; fungi such as Candida, actinomycetes, geotrichum, Aspergillus, Cryptococcus, and Nocardia and the like; bacteria such as Mycoplasma, Legionella, Haemophilus, Pneumococcus and Bordetella pertussis. Diseases caused by nosocomial infection also can be cited among such infectious disease.

For example, as shown in FIG. 1, the system 100 includes sensors 60 provided in each area for a plurality of areas arranged in the facility. The sensor 60 measures the environmental parameters 50 in the area related to the risk of occurrence of disease. The system 100 also includes a display unit 32 for displaying the in-facility area layout diagram (also referred to as a facility map) such as a floor map M301 showing the positions of the plurality of areas.

The system 100 is provided with a control unit 21a which generates display data (caution alert display data) 40 for identifiably displaying the environmental condition of each area on the in-facility area layout diagram based on the measured value of the environmental parameter 50 output from the sensors 60. The caution alert display data 40 includes, for example, caution alerts C 221 and C 222 for displaying the area in which the environmental condition is sub-nominal on the floor map M301.

In the floor map M301 exemplified in FIG. 1, the caution alert C221 is displayed in the area with the area number “7101”, and the caution alert C222 is displayed in the area with the area number “7102” and the area with the area number “7115”. The caution alert C221 is an icon including two exclamation points surrounded by a circular frame indicating the environmental condition has extremely deteriorated and the caution call level is high, that is, this is a “warning” that the risk of occurrence of a disease is extremely high. The caution alert C221 also indicates that the disease is influenza. The indication of this disease is optional. The caution alert C222 is an icon including one exclamation point surrounded by a circular frame indicating that the environmental condition has relatively deteriorated and that the caution call level is low, that is, this is a “caution”. The notice C222 also indicates that the disease is heat stroke. The indication of this disease is optional. Caution alerts C221 and C222 are not displayed in the areas with a nominal environmental condition.

For example, by confirming the caution alerts C221 and C222 in the floor map M 301 of FIG. 1 displayed on the display unit 32 arranged in the nurse station, for example, a nurse reliably comprehends the areas in which the environmental condition is sub-nominal. Therefore, the nurse can promptly take measures to improve the environmental condition of the area in which the environmental condition is sub-nominal.

For example, in the example shown in FIG. 1, it can be readily comprehended that the environmental conditions of the areas with the area numbers “7101”, “7102” and “7115” are sub-nominal. Therefore, the nurse can promptly implement countermeasures such as additionally operating a humidifier or a dehumidifier in these areas, adjusting the set temperature of the air conditioner, actively ventilating by operating the ventilator and the like.

Here, the facility 900 is not limited insofar as it can accommodate a plurality of people. For example, the facility 900 may be a medical facility such as a hospital, an educational facility such as a school, a welfare facility such as a day care center, a nursery school, a kindergarten, a senior center and the like.

The area also may be a building (unit) provided in the facility 900. The area also may be a space such as a room separated from the surrounding space by a wall, a partition and/or a door in the building, or may be a hallway, a space or the like which is partially open. The area also may be around a bed used by one patient in each room (as a guide, for example, a range of a curtain stretched around the bed at bedtime and the like). For example, when the facility 900 is a medical facility, a ward, a ward floor, a hospital room, and the like can be cited as an area.

### System Configuration

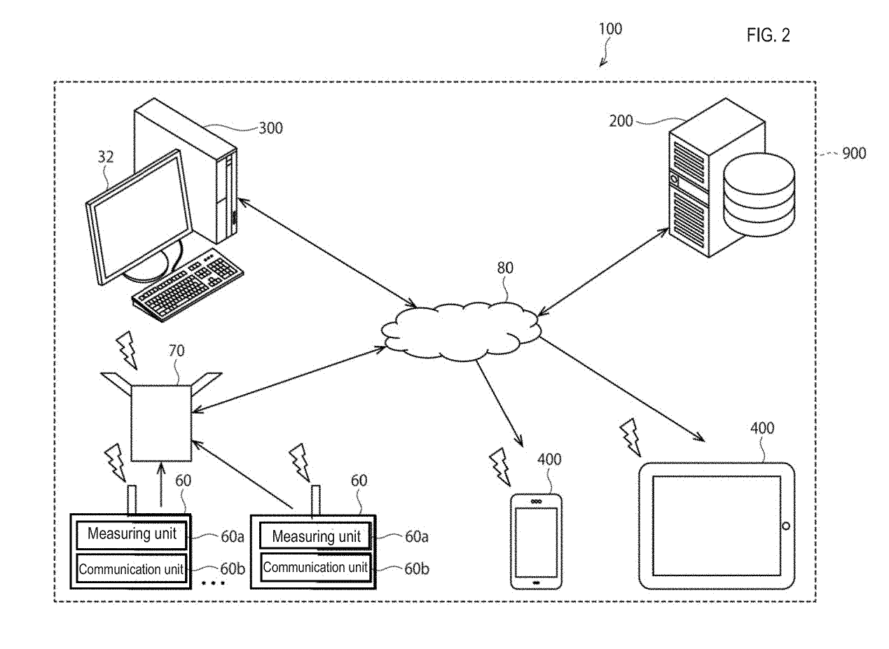

Hereinafter, using the medical facility as an example, the configuration of the system 100 will be described with reference to FIG. 2. However, embodiments of the present invention are not to be construed as limited to medical facilities. The system 100 includes at least a sensor 60 provided in each area, an in-facility monitoring apparatus 200 (hereinafter also referred to as a monitoring apparatus 200) having a control unit (CPU) 21a and a display unit 32. The display unit 32 is connected to the client device 300. In the system 100, the monitoring apparatus 200 and the client device 300 are connected to each other via a communication network 80.

The sensor 60 is provided in each area of the facility 900. Although the sensor 60 can be provided at any location in each area, it may be disposed in every ward, every room or bed of a hospitalized patient and preferably at the side of the bed on the patient's head side. The sensor 60 has a communication unit 60b. The communication unit 60b of the sensor 60 is connected to a wireless device 70 such as a Wi-Fi (registered trademark) router and further connected to the communication network 80. The communication network 80 may be a wired network or a wireless network. The sensor 60 provided in each area transmits the value of the measured environmental parameter 50 to the monitoring apparatus 200.

The monitoring apparatus 200 determines the environmental condition for each area based on the measured value of the environmental parameter 50 collected from the sensor 60, and transmits the determination result to the client apparatus 300. The monitoring apparatus 200 may be installed in the facility, or it may be installed outside the facility.

The client device 300 is used by a medical worker (hereinafter referred to as a user) such as a doctor and a nurse, for example, and is installed in a nurse station or the like. The client device 300 displays the determination result of the environmental condition transmitted from the monitoring apparatus 200 together with the area layout map in the facility (for example, the hospital room map M301 shown in FIG. 1) on the display unit 32.

The system 100 also may include a mobile terminal 400 such as a smart-phone, a tablet terminal, a laptop computer or the like. The mobile terminal 400 is mainly used by the user in the facility 900 and is notified of the determination result of the environmental condition transmitted from the monitoring apparatus 200. The mobile terminal 400 also can display the layout diagram of the area within the facility, together with the determination result.

### Hardware Configuration

### In-Facility Monitoring Apparatus

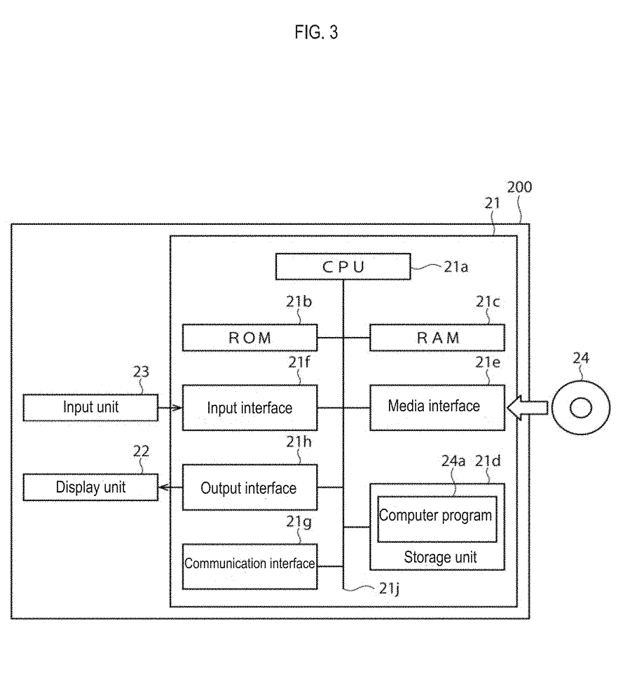

As shown in FIG. 3, the monitoring apparatus 200 is realized by, for example, a general-purpose computer. The monitoring apparatus 200 includes a main body 21, a display unit 22, and an input unit 23. The display unit 22 and the input unit 23 have optional configurations. The main body 21 includes a CPU (Central Processing Unit) 21a, a ROM (read only memory) 21b, a RAM (Random Access Memory) 21c, a storage unit 21d, a media interface 21e, an input interface 21f, a communication interface 21g, and an output interface 21h. The CPU 21a, the ROM 21b, the RAM 21c, the storage unit 21d, the media interface 21e, the input interface 21f, the communication interface 21g, and the output interface 21h are connected by a bus 21j. The storage unit 21d is realized by a hard disk, an SSD or the like.

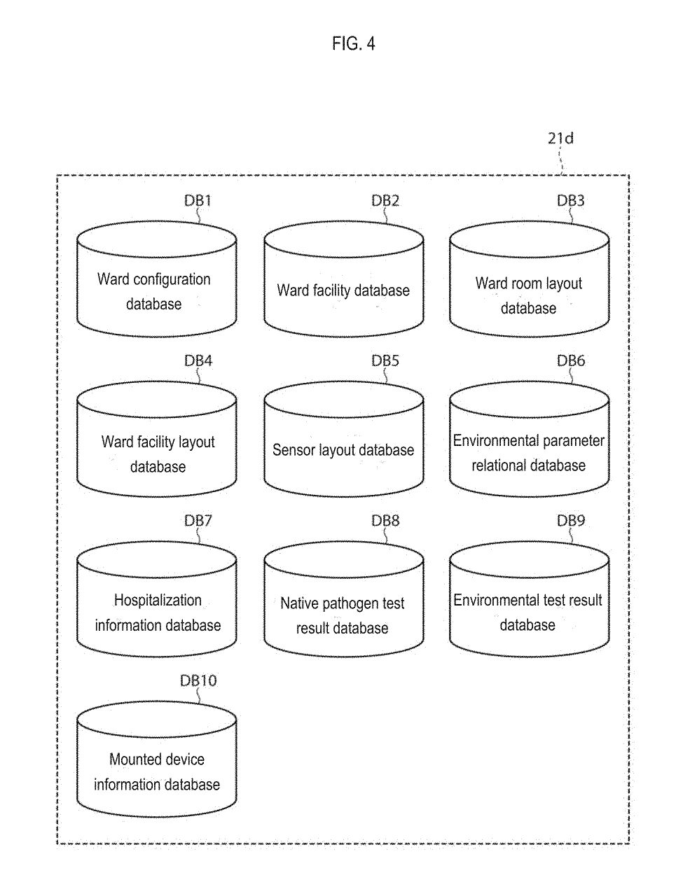

Various programs such as an operating system and application programs, various computer programs to be executed by the CPU 21a, and data used for executing a computer program, are installed in the storage unit 21d. Various databases shown in FIG. 4 to be described later also are stored. A computer program 24a for operation of the monitoring apparatus 200 described later is also installed in the storage unit 21d.

The media interface 21e is connected to, for example, a CD-ROM drive, a DVD-ROM drive or the like (not shown) and can read a computer program or data recorded on a portable recording medium 24 such as a removable medium. The portable recording medium 24 is a tangible recording medium that is computer readable and is not temporary, and the portable recording medium 24 stores a computer program 24a for causing the computer to function as the monitoring apparatus 200. The monitoring apparatus 200 can read the computer program 24a from the portable recording medium 24, and install the computer program 24a in the storage unit 21d. The CPU 21a functions as a control unit by reading the computer program 24a in the RAM 21c and performing various arithmetic processes. Note that the monitoring apparatus 200 also may download the computer program 24a via a communication network such as the Internet.

In the storage unit 21d, for example, a multitask operating system such as Windows (registered trademark) manufactured and sold by Microsoft Corporation of USA is installed. In the following description, the computer program 24a according to the invention operates on the operating system. The configuration of various databases provided in the storage unit 21d will be described later with reference to FIG. 4.

The input interface 21f is configured by, for example, a serial interface such as USB, IEEE 1394, or RS-232 C, a parallel interface such as SCSI, IDE, or IEEE 1284, and an analog interface configured by a D/A converter and A/D converter. An input unit 23 such as a keyboard and a mouse can be connected to the input interface 21f.

The communication interface 21g is, for example, an Ethernet (registered trademark) interface. The communication interface 21g is connected to the client device 300, the sensors 60, and the mobile terminal 400 via the communication networks 80 and 90. The monitoring apparatus 200 can transmit and receive data to and from the client device 300, the sensors 60, and the mobile terminal 400 connected to the communication networks 80 and 90 by using the communication interface 21g using a predetermined communication protocol.

The output interface 21h also may have the same configuration as the input interface 21f. The output interface 21h is connected to a display unit 22 composed of a liquid crystal display or the like, and outputs a video signal corresponding to the image data output from the CPU 21a to the display unit 22. The display unit 22 displays a screen according to the input video signal.

As shown in FIG. 4, the storage unit 21d is provided with a ward configuration database DB1, a ward facility database DB2, a ward room layout database DB3, a ward facility layout database DB4, a sensor layout database DB5, and an environment parameter relational database DB6. The storage unit 21d also may store the hospitalization information database DB7, the native pathogen test result database DB8, the environmental test result database DB9, the mounted device information database DB10 and the like.

Information on the ward floor, hospital room, bed and the like provided in each ward is stored in the ward configuration database DB1. Specifically, for example, the management number, the hospital code, hospital ward name, floor name, hospital room number, hospital room name, and the bed number are stored in the ward configuration database DB1.

Information on facilities (for example, toilets, elevators, stairways, bathrooms, washrooms and the like) other than a room in the ward is stored in the ward facility database DB2. Specifically, for example, the management number, the hospital ward code, the hospital room number, the facility code assigned for each facility, and the facility name are stored in the ward facility database DB2.

Layout information of each hospital room in the ward is stored in the hospital room layout database DB3. Specifically, for example, the management number, the hospital ward code, the hospital room number, and the coordinates of the hospital room are stored in the hospital room layout database DB3. Hospital room layout information stored in the hospital room layout database DB3 is used when screen data for facility map display described later are created.

Layout information of each facility in the ward is stored in the ward facility layout database DB4. Specifically, for example, the management number, the hospital ward code, the facility code, and the coordinates of the facility are stored in the ward facility layout database DB4. The facility layout information stored in the ward facility layout database DB4 is used when screen data for facility map display described later are created.

Layout information of each sensor 60 in the ward is stored in the sensor layout database DB5. Specifically, for example, the management number, the hospital ward code, the hospital room number, the sensor identification code, and the coordinates of the sensor are stored in the sensor layout database DB5. The sensor layout information stored in the sensor layout database DB5 is used when screen data for facility map display which will be described later are created.

The measurement values of the sensors 60 arranged in the ward are stored in the environmental parameter relational database DB6. Specifically, for example, the sensor identification code, the measurement value of each environmental parameter 50, and the measurement time are stored in the environment parameter relational database DB6. A reference range corresponding to the measurement value of each environmental parameter 50 and a reference range corresponding to the index obtained from the measurement value of the environmental parameter 50 are stored in the environmental parameter relational database. The sensor information stored in the environment parameter relational database DB6 is used in a caution alert display process to be described later.

Information related to hospital admission and discharge of a patient is stored in the hospitalization information database DB7. The hospitalization information database DB7 stores hospitalization information included in medical information transmitted from the electronic medical record system of the target medical facility. Specifically, the patient ID, the name of the patient, the admission date, the discharge date, the moving date, the hospital code of the ward where the patient is hospitalized, the name of the hospital ward, the room number of the hospital where the patient is hospitalized and the like are stored in the hospitalization information database DB7. The hospitalization information stored in the hospitalization information database DB7 is used when creating screen data for displaying the facility map, which will be described later.

The native pathogens test result database DB8 stores the result of the pathogen test of the patient. Native pathogen test results transmitted from a sample analyzer are stored n the native pathogen test result database DB8. Specifically, information such as the patient ID, the name of the patient, the sample collection date and time, the pathogen code indicating the detected pathogen, and the name of the pathogen are stored in the native pathogen test result database DB8. Test results of infectious diseases obtained by examinations other than by the sample analyzer (for example, test results of influenza antigen A, enteric infectious virus, cold and the like) are acquired from the electronic medical record system of the targeted medical facility. The result of examination of infectious diseases is also stored in the native pathogen test result database DB8. The native pathogen test result stored in the native pathogen test result database DB8 is used when creating screen data for facility map display which will be described later.

The result of the environmental test carried out at the target medical facility is stored in the environmental test result database DB9. The environmental test is an inspection for detecting bacteria or the like as a pathogen from a disease room or equipment contaminated with a pathogen. Specifically, the control number, hospital room number, facility code, environmental examination method, pathogen code, and name of the detected bacteria are stored in the environmental examination result database DB9. The environmental examination result stored in the environmental test result database DB9 is used when creating screen data for displaying the facility map, which will be described later.

Information related to a device worn by the patient is stored in the attached device information database DB10. The term “device” as used herein means a medical device to be worn on a patient, and examples thereof include a catheter, a drain, an infusion route, an ED tube, an ileus tube, a ventilator, and the like. The monitoring apparatus 200 communicates with the electronic medical chart system of the targeted medical facility and receives medical care information of the patient stored in the electronic medical record system. Information on a device worn by a patient, which is included in medical information transmitted from the electronic medical record system, is stored in the attached device information database DB10. Specifically, a patient ID for identifying a patient, a device insertion/removal date, a device code assigned to a device for which insertion and removal was performed, a device name, and a hospital code indicating a ward where a patient is hospitalized and the like are stored in the attached device information database DB10. The attached device information stored in the attached device information database DB10 is used when creating screen data for displaying a facility map, which will be described later. The monitoring apparatus 200 also may have a role, for example, as a so-called server. In this case, the CPU 21a controls the monitoring apparatus 200 using a server operating system (OS) such as Linux (registered trademark), UNIX (registered trademark), Microsoft Windows Server (registered trademark), or the like.

### Client Device

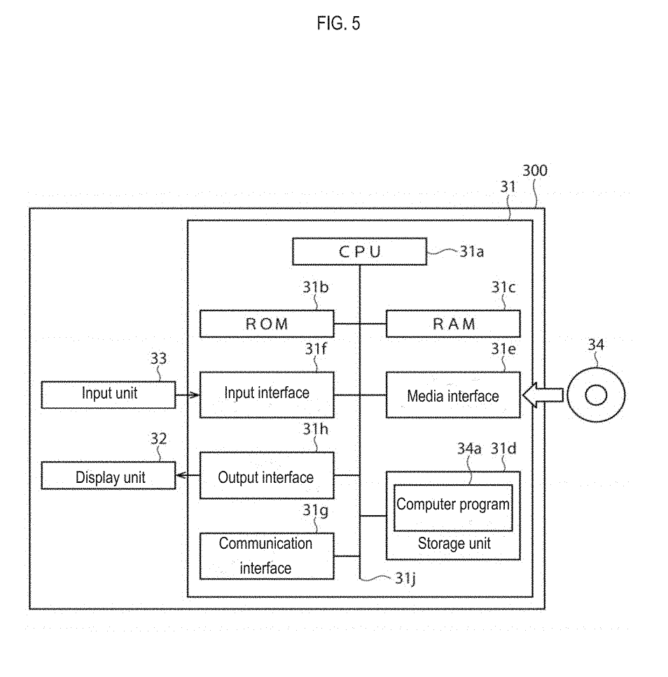

As shown in FIG. 5, the client device 300 is realized by, for example, a general-purpose computer. The client apparatus 300 includes a main body 31, a display unit 32, and an input unit 33. The display unit 32 and the input unit 33 may be integrated and realized as a touch panel type display unit. The main body 31 includes a CPU 31a, a ROM 31b, a RAM 31c, a storage unit 31d, a media interface 31e, an input interface 31f, a communication interface 31g, and an output interface 31h. The CPU 31a, the ROM 31b, the RAM 31c, the hard disk 31d, the media interface 31e, the input interface 31f, the communication interface 31g, and the image output interface 31h are connected by a bus 31j. The storage unit 31d is realized by a hard disk, an SSD, or the like.

Various computer programs to be executed by the CPU 31a, such as an operating system and an application programs, and data used for executing a computer program, are installed in the storage unit 31d.

The media interface 31e is connected to, for example, a CD-ROM drive, a DVD-ROM drive or the like (not shown), and can read a computer program or data recorded on a portable recording medium 34 such as a removable medium. The portable recording medium 34 is a computer readable and non-transitory tangible recording medium, and the portable recording medium 34 stores a computer program 34a for causing a computer to function as the client device 300. The client device 300 can read the computer program 34a from the portable recording medium 34 and install the computer program 34a in the storage unit 31d. Alternatively, the client device 300 may download the computer program 34a via a communication network such as the Internet.

For example, a multitasking operating system such as Windows (registered trademark) manufactured and sold by Microsoft Corporation of USA is installed in the storage unit 31d. The computer program 34a operates on the operating system.

Other configurations of the client device 300 are similar to the configuration of the monitoring apparatus 200 described above, so the description is hereby incorporated by reference.

The client device also may be the same as the monitoring apparatus 200.

### Mobile Terminal

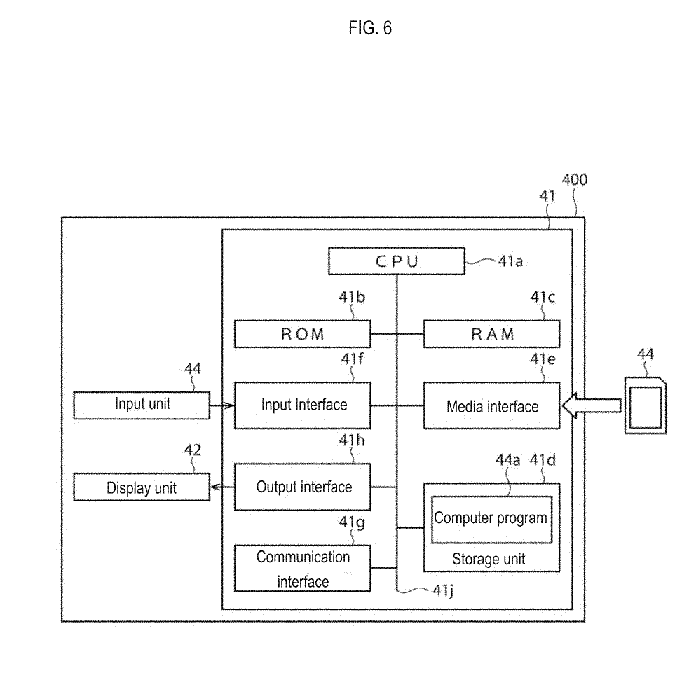

As shown in FIG. 6, the mobile terminal 400 is realized by a computer such as a smart-phone, a tablet device, a laptop computer, or the like. The mobile terminal 400 includes a main body 41, a display unit 42, and an input unit 43. It is preferable that the display unit 42 and the input unit 43 are integrated and realized as a touch panel type display unit. The main body 41 includes a CPU 41a, a ROM 41b, a RAM 41c, a storage unit 41d, a media interface 41e, an input interface 41f, a communication interface 41g, and an image output interface 41h. The CPU 41a, the ROM 41b, the RAM 41c, the hard disk 41d, the media interface 41e, the input interface 41f, the communication interface 41g, and the image output interface 41h are connected by a bus 41j. The storage unit 41d is realized by a hard disk, an SSD, or the like.

Various computer programs to be executed by the CPU 41a, such as an operating system and application programs, and data used for executing a computer program, are installed in the storage unit 41d.

The media interface 31e is connected to, for example, an SD drive or the like (not shown), and can read a computer program or data recorded on a portable recording medium 44 such as a removable medium. The portable recording medium 44 is a tangible recording medium that is computer readable and is not temporary, and a computer program 44a for causing a computer to function as the mobile terminal 400 is stored in the portable recording medium 44. The mobile terminal 400 can read the computer program 44a from the portable recording medium 44 and install the computer program 44a in the storage unit 41d. Alternatively, the mobile terminal 400 also may download the computer program 44a via a communication network such as the Internet.

An operating system such as iOS (registered trademark) manufactured and sold by Apple Co., Ltd. and Android (registered trademark) manufactured and sold by Google Inc., for example, is installed in the storage unit 41d. The computer program 44a operates on the operating system.

Other configurations of the portable terminal 400 are the same as those of the above-described client device 300, so the description thereof is incorporated herein.

### Sensors

As shown in FIG. 2, the sensors 60 includes at least a measuring unit 60a for measuring environmental parameters and a communication unit 60b for communicating with the wireless device 70. The measuring unit 60a is not limited insofar as it can quantitatively or semi-quantitatively measure the information of the environmental factor. The information of the environmental factor quantitatively or semi-quantitatively measured by the measuring unit 60a is also referred to as measurement information of the environmental factor. Specifically, the measurement unit 60a can quantitatively or semi-quantitatively measure the temperature information, the humidity information, the ventilation information, and/or the light information to obtain the measurement information of each environmental factor. Here, “information” is a numerical value indicating the value of each parameter (Celsius (or Fahrenheit) in case of temperature information, relative humidity in case of humidity information, light intensity in case of light information, CO₂concentration in the case of ventilation information and the like), a voltage or the like output from each element of the measuring unit (a temperature sensing element in the case of temperature information, a moisture sensing element in the case of humidity information, a photosensitive element in case of optical information, or a gas sensing element in the case of ventilation information and the like). Although the sensor 60 may measure temperature, humidity, CO₂ concentration, and light separately, they also may be measured at the same time.

The communication unit 60b is configured by communication modules such as LPWA (Low Power Wide Area), 3G, LTE (Long Term Evolution), BLE (Bluetooth (registered trademark) Low Energy), for example. The communication unit 60b may directly connect to the communication network 80.

Known sensors can be used as the sensors, such as SkyLogger from SKYDISK Co., Ltd., and the like.

Preferably, the sensor 60 measures the environmental parameter at predetermined time intervals, and transmits the measured value of the environmental parameter together with the time of measurement to the monitoring apparatus 200 via the communication network 80. The predetermined time interval is, for example, 10 minutes. The monitoring apparatus 200 records the received measurement value and measurement time information in association with the sensor identification code in the environment parameter relational database DB6.

### System Operation

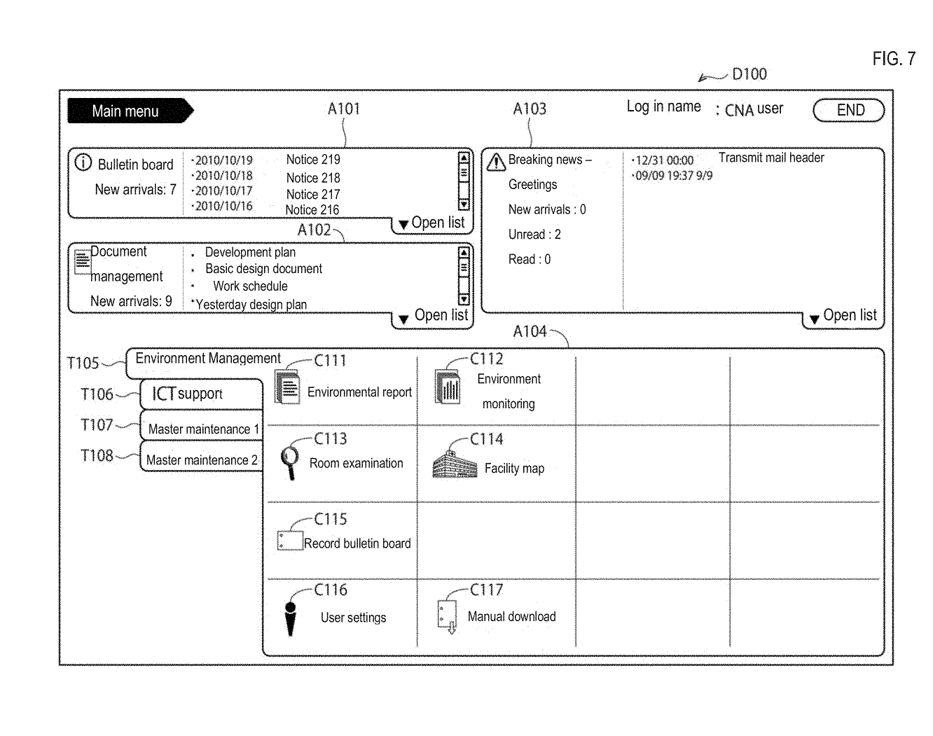

In the system 100 according to the embodiment of the invention, operation is started by the user operating the input unit 33, for example, and activating the system 100. It also is possible for a plurality of users to log into the system 100 at the same time or at different times. The user operates the input unit 33 of the client device 300 to input a user name and a password, for example, to make a request to log into the in-facility monitoring system 100. The monitoring apparatus 200 receives the user name and password and performs login authentication. When the login authentication succeeds, for example, the client device 300 displays the menu screen D100 shown in FIG. 7 on the display unit 32.

### Main Menu Screen

As shown in FIG. 7, a bulletin board area A101, a document management area A102, a mail operation area A103, and a menu area A104 are provided on the main menu screen D100. The bulletin board area A101 is used for communication between users. The document management area A102 is used for browsing, creating, editing, saving, deleting of the document data relating to the environment parameter 50. The mail operation area A103 is used for viewing, creating, sending of mails. The menu area A104 is used to switch the display to each screen of the system 100.

A plurality of menus for calling various functions of the system 100 are provided in the menu area A104. Four tabs T105, T106, T107, and T108 are provided n the menu area A104. When the user selects one of these tabs T105, T106, T107, and T108 by a mouse operation or the like, an icon corresponding to the selected tab is displayed in the menu area A104.

The icons included in the environmental management tab T105 are assigned functions used for management of the environment in the hospital. Specifically, the environmental management tab T105 includes an environmental report icon C111, an environmental monitoring icon C112, a patient search icon C113, a facility map icon C114, a bulletin board registration icon C115, a user setting icon C116, a manual download icon C117.

The environmental report icon C111 is assigned the display function and creation function of the environmental report. The environmental monitoring icon C112 is assigned a display function of the measured values of each environmental parameter 50 and/or indices obtained from measured values of each environmental parameter 50. The measured values or index is displayed in real time or user-specified time, every weekday or daily, every day of the month, or weekly. The display function of the environmental condition for each ward or floor is assigned to the facility map icon C114. The information posting function to the bulletin board is assigned to the bulletin board registration icon C115. The user setting icon C116 is assigned a function of setting information on a user who uses the system 100. A function for downloading an operation manual of the system 100 is assigned to the manual download icon C117.

The term “icon” as used herein means an image assigned to a specific function and designed so as to symbolically represent the assigned function, and also includes an image to be displayed in the window.

The function assigned to each icon of the environmental management tab T105 is used by an infection control doctor (ICD) and an infection control nurse (ICN) to take countermeasures against infectious diseases. the functions assigned to each icon of the environmental management tab T10 is also used by medical staff such as a doctor and a nurse who perform a medical treatment at an ordinary medical site such as a hospital room.

The ICT support tab T106 is provided with an icon (not shown) to which a function for supporting the infection control team (ICT) is assigned. The infection control team is a team composed of infection control doctors and infection control nurses who are experts in infection control.

Each of the master maintenance tabs T107 and T108 is provided with an icon (not shown) for master management of various databases used in the system 100. The various databases are the ward configuration database DB1, the ward facility database DB2, the ward room layout database DB3, the ward facility layout database DB4, the sensor layout database DB5, the environment parameter relational database DB6 provided in the storage unit 21d of the monitoring apparatus 200.

When the facility map icon C114 is selected on the menu screen D100, a map display process for displaying a map in the facility described below and a caution alert display process for displaying a caution alert on the in-facility map are transmitted to, and jointly executed on, the monitoring apparatus 200 and the client device 300. The display contents of the facility map and the display contents of the caution alert can be automatically updated at predetermined time intervals, for example, by the client device 300 automatically calling up the map display function and the caution alert display function. Alternatively, the display content of the facility map and the display content of the caution alert may be manually updated by the user selecting the icon and calling up the map display function and the caution alert display function.

### Map Display Process 1

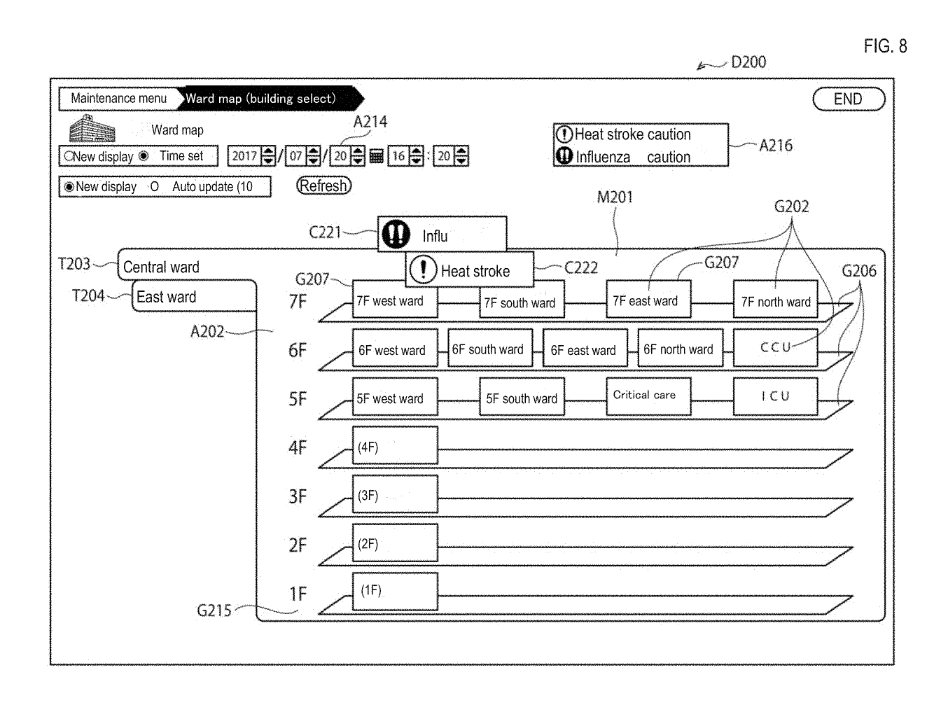

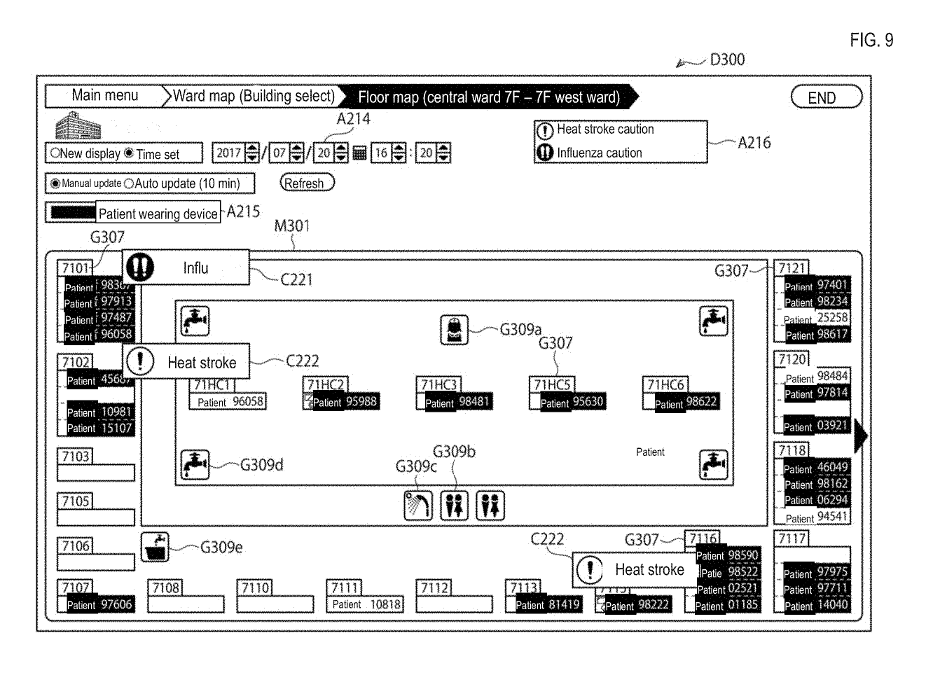

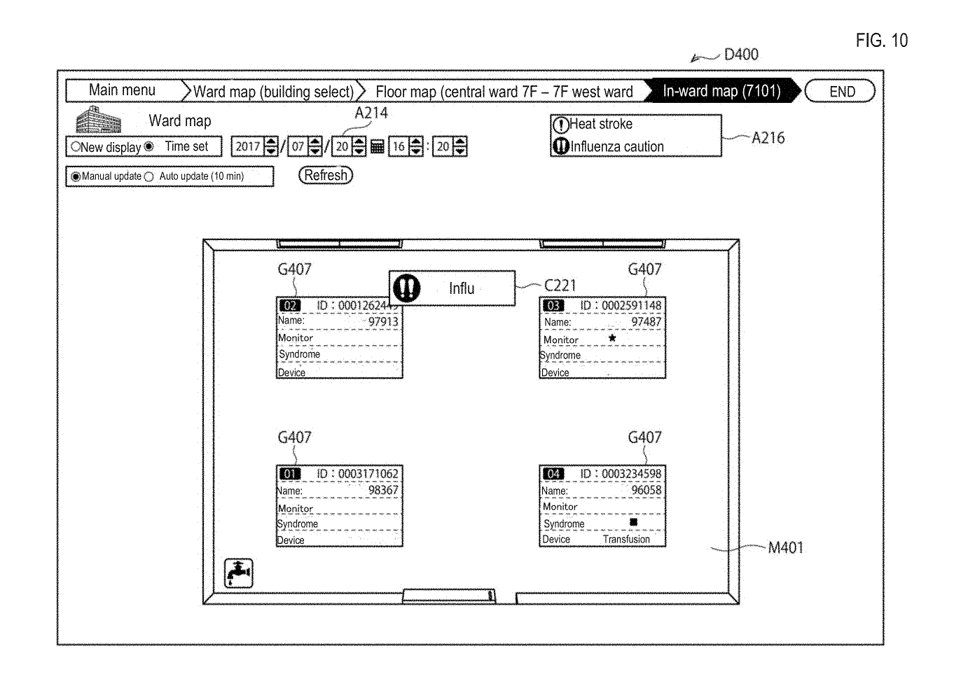

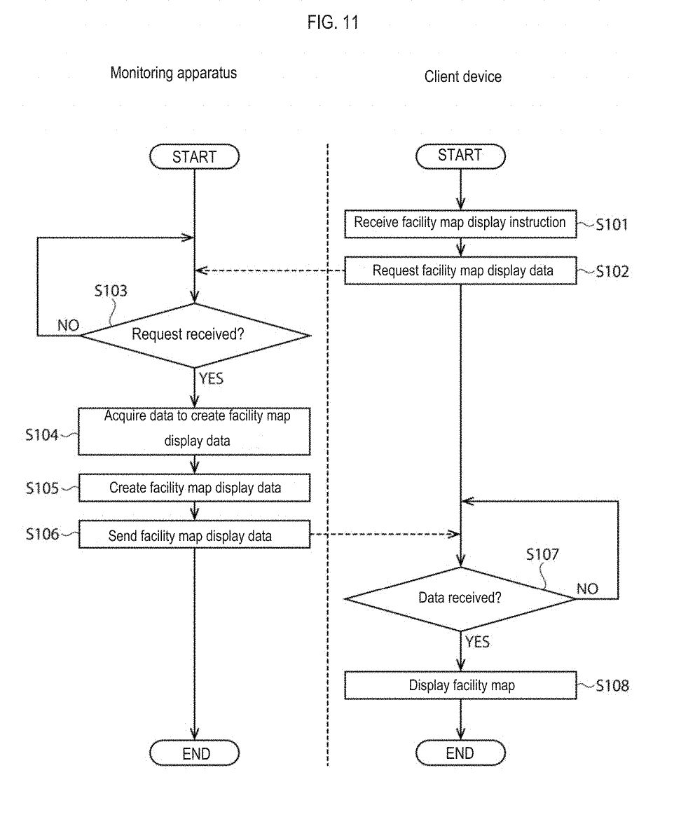

In the map display process 1, when the facility map icon C114 displayed on the main menu is selected on the client device 300, for example, the ward map screen D200 (FIG. 8) including the ward configuration map M201, the floor map screen D300 (FIG. 9) including the floor map M301, and the patient room map screen D400 (FIG. 10) including the patient room map M401 are displayed on the display unit 32 or the like of the client device 300. These three maps may be displayed in parallel on the same screen when the facility map icon C114 is selected. When the facility map icon C114 is selected, the ward map M201, the floor map M301, and the patient room map M401 also may be displayed in three levels in order from the upper layer to the lower layer. When the facility map icon C114 is selected on the main menu screen, the monitoring apparatus 200 displays the facility map (for example, the ward map M201, the floor map M301, and the patient room map M401) to the client device 300 on the display unit 32 in sequence.

In the facility map, information related to each area stored in the ward building database DB1, the ward facility database DB2, the room layout database DB3, the ward facility layout database DB4 and the like, personal information related to people staying in each area (for example, patient information such as age, gender, hospitalization date), equipment used by the person (for example, a catheter, a blood transfusion route, a ventilator and the like), and area information such as disease information from which the person is suffering is displayed.

The flow of the facility map display processing in the system 100 will be described with reference to the flowchart shown in FIG. 11. In step S101, the facility map icon C114 is selected by the input from the user's input unit 33, and the CPU 31a of the client device 300 receives the facility map display instruction.

In step S102, the CPU 31a of the client device 300 transmits a request instruction for requesting the facility map display data necessary for displaying the facility map on the monitoring apparatus 200 via the communication interface 31g of the client device 300. The request instruction transmitted from the client device 300 is received by the monitoring apparatus 200 via the communication interface 21g.

Instead of the facility map icon C114, icons for selecting one facility map of the ward configuration map M201, the floor map M301, and the patient room map M401 may be displayed in the main menu. When the facility map to be displayed is the ward map M201, the transmission instruction may include information (for example, a name such as “central ward”) for specifying the ward where display is requested. When the facility map to be displayed is the floor map M301, the request instruction may include information (for example, a ward code) specifying a floor to be displayed. When the facility map to be displayed is the section map M401, the request instruction may include information (for example, a ward room number) for specifying an area to be displayed.

In step S103, the CPU 21a of the monitoring apparatus 200 waits until it receives a request instruction.

When the monitoring apparatus 200 receives the request instruction, in step S104, the CPU 21a acquires the data necessary to display the facility map from the facility map from the ward configuration database DB1, the ward facility database DB2, the patient room layout database DB3, the ward facility layout database DB4, and the sensor layout database DB5.

In step S105, the CPU 21a creates facility map display data for the facility map requested based on the acquired information. The created facility map display data may be temporarily stored in the RAM 21c or may be stored in the storage unit 21d.

In step S106, the CPU 21a transmits the created facility map display data to the requesting client device 300 via the communication interface 21g. The facility map display data transmitted from the monitoring apparatus 200 is received by the client device 300 via the communication interface 31g.

In step S107, the CPU 31a of the client device 300 waits until it receives facility map display data.

When the client device 300 receives the facility map display data, in step S108 the CPU 31a displays the facility map screen D200, D300, or D400 shown in FIG. 8 to FIG. 10 on the display unit 32 based on the received facility map display data.

The facility map screen D200 illustrated in FIG. 8 includes the ward map M201. The ward map M201 displays the structure of the entire ward as a simplified figure. The facility map screen D300 illustrated in FIG. 9 includes a floor map M301. The floor map M301 is a map created for each floor of each ward, and displays the structure of the entire floor as a simplified figure. The facility map screen D400 exemplified in FIG. 10 includes a patient room map M401. The patient room map M401 is a map created for each area, and displays the structure of the entire interior in a simplified figure.

### Ward Map

Referring to FIG. 8, the ward map screen D200 is provided with a ward map area A202 on which the ward map M201 is displayed. In the embodiment, since a plurality of wards are provided in the medical facility, the ward switching tabs T203 and T204 are displayed in the ward map area A202. The ward switching tabs T203 and T204 can be selected by a user's mouse click operation or the like, and the ward map M201 of the ward corresponding to the selected tab is displayed in the ward map area A202.

The ward map M201 displays the structure of the entire ward as a simplified figure. In the embodiment, since the ward is composed of a plurality of floors, a ward map M201 on which floors on each floor are shown is displayed. A floor number display G215 is attached on the left side of the floor view G206 of each floor. In the floor view G206, floor information G207 is displayed for each floor. Although not shown, the floor information G207 may include the name of each floor and the number of patients hospitalized on the floor. In this way it is easy to comprehend which hospitalized patients are on which floor. When a plurality of floors are provided on the same floor, floor information G207 is displayed for each floor.

For example, in the example shown in FIG. 8, the seventh floor is divided into four floors of “7th floor west”, “7th floor south”, “7th floor east” and “7th floor north”. For example, the floor information F207 on the “7th floor west” floor includes the floor name G202 “7th floor west ward”. Floor information F207 on the “7th floor south” floor includes the floor name G202 “7th floor south ward”. Floor information F207 on the “7th floor east” floor includes the floor name G202 “7th floor eastern ward”. Floor information F 207 on the “7th floor north” floor includes the name G202 “7th floor north ward” of the floor.

Caution alerts C221 and C222 on the floor also are displayed in the floor diagram G206 on each floor. The caution alerts C221 and C222 are indicated by icons including exclamation points surrounded by, for example, a circular frame based on a result of a caution alert display process to be described later. The number of exclamation marks corresponds to the caution call level, which means that the greater number of exclamation points, the higher the caution level. The number of stages of the caution call level is not particularly limited, but in this embodiment it is two stages. For example, when there is at least one area on each floor corresponding to the floor information G207 in which it is determined that a caution alert should be issued by the caution alert display process, caution alerts C221 and C222 of types corresponding to the determination result are displayed beside the corresponding floor information G207.

The date and time designation area A214 includes an input box for specifying the date and time. By specifying the date and time in the input box, the display is updated in the ward map showing the infection status in the ward at the designated date and time.

The caution alert legend area A216 is an area in which an explanation about the level of caution indicated in the caution alerts C221 and C222, and is displayed to explain the caution level.

### Floor Map

Each floor information G207 can be selected by the user clicking the mouse. When one floor information G207 is selected, the facility map display process shown in FIG. 11 is called up, and a floor map M301 of the selected floor is displayed.

In the example shown in FIG. 9, a floor map M301 is displayed on the floor map screen D300. A date and time designation area A214 for specifying the date and time, and a caution alert legend area A216 are provided above the floor map M301. The date and time designation area A214 and the caution alert legend area A216 provided on the floor map screen D300 are the same as the date and time designation area A214 and the caution alert legend area A216 provided in the ward map screen D200 shown in FIG. 8 . A legend area A215 indicating whether the patient is wearing a device may be provided under the legend area A214.

The floor map M301 shown in FIG. 9 displays the structure of the entire floor as a simplified figure. In the floor map M301, the area information G307 is displayed for each area accommodating a patient. The area information G307 includes the area number of each area and the patient ID of the patients hospitalized in the area. When the patient is wearing a device such as a ventilator, a catheter, a blood transfusion route or the like, the patient ID is displayed in, for example, black and white inversion in the area information G307. For example, all four patients hospitalized in the room number “7101” are wearing some medical equipment (device) such as a ventilator, a catheter, and a transfusion route.

In the floor map M301, caution alerts C221 and C222 are displayed for each area corresponding to the area information G307. The caution alerts C221 and C222 are indicated by exclamation marks surrounded by, for example, a circular frame based on the result of the caution alert display process described later. For example, when there is at least one sensor 60 to issue a caution alert via the caution alert display process in each area corresponding to the area information G307, the type of caution alert C221 and C222 corresponding to the determination result is displayed in the vicinity of the corresponding area information G307.

In the floor map M301, facility information such as, for example, the nurse station G309a, the toilet G309b, the bathroom G309c, the washroom G309d, the soiled item disposal room G309e and the like are displayed as facilities outside the rooms of the patients. Here, the area includes facilities other than the hospital room, as well as the hospital rooms in the ward.

### Patient Room Map

The user selects the area information G307 of the area desired to be displayed out of the area information G307 of the floor map M301 shown in FIG. 9 to call up the map display process shown in FIG. 11, and displays the patient room map M401 of the selected room.

In the example shown in FIG. 10, the patient room map M401 is displayed on the patient room map screen D400.

The patient room map M401 displays the entire structure of the room as a simplified figure. In the patient room map M401, information on the person staying in the room, for example, the patient identification number (ID), the name of the patient, the infectious disease of the patient, symptoms (syndrome) presented by the patient and the like are displayed for each patient bed. When the patient wears a device, the type of the device worn by the patient is displayed in the “device” column of the hospitalized patient information G407. The sensor 60 is arranged at each bed of a patient, and a caution notice C221 is displayed for each bed corresponding to the hospital patient information G407 in the patient room map M401.

Here, steps S103 to S106 are not necessarily essential in the operation of the system 100, and the facility map display data generated in the steps S103 to S106 may be stored in the storage unit 31d of the client device 300 or the storage unit 21d of the monitoring apparatus 200. Then, the CPU 31a of the client device 300 may call up the facility map display data from the storage unit 31d or the storage unit 21d according to a request instruction in step S102.

### Map Display Process 2

In this embodiment, in addition to the facility map, information relating to a patient having a native a pathogen (referred to as a pathogen carrier) that may infect another patient, the type of the pathogen, presence or absence of a pathogen in the environment and the like is displayed. By performing such a display, the medical staff can use not only the state of the indoor environment but also consider the pathogen already native to the patient, the device already worn by the patient and the like to achieve comprehensive management and determinations aimed at preventing nosocomial infection. In particular, from the display of the facility map, it becomes possible to easily identify the room in which a seriously ill patient is hospitalized, so that management and judgment can be performed in consideration of the corresponding priority.

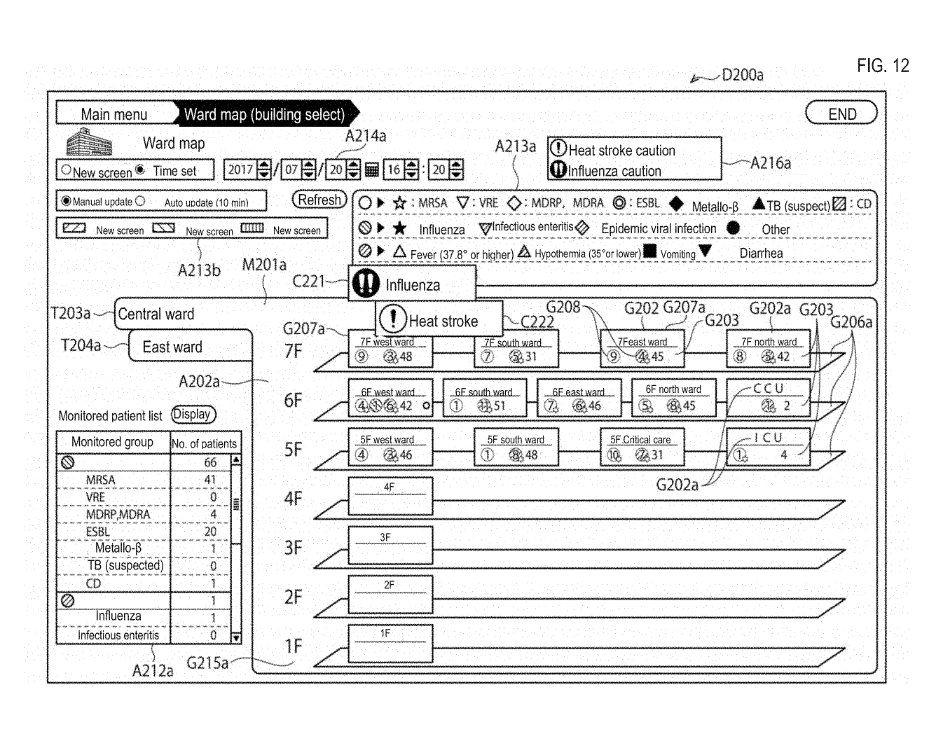

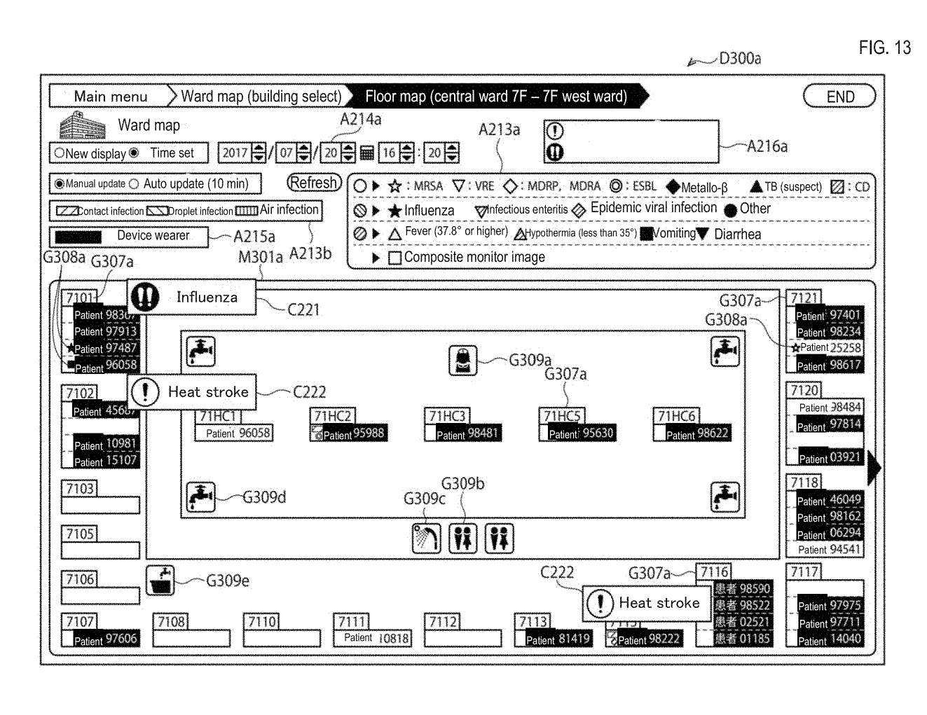

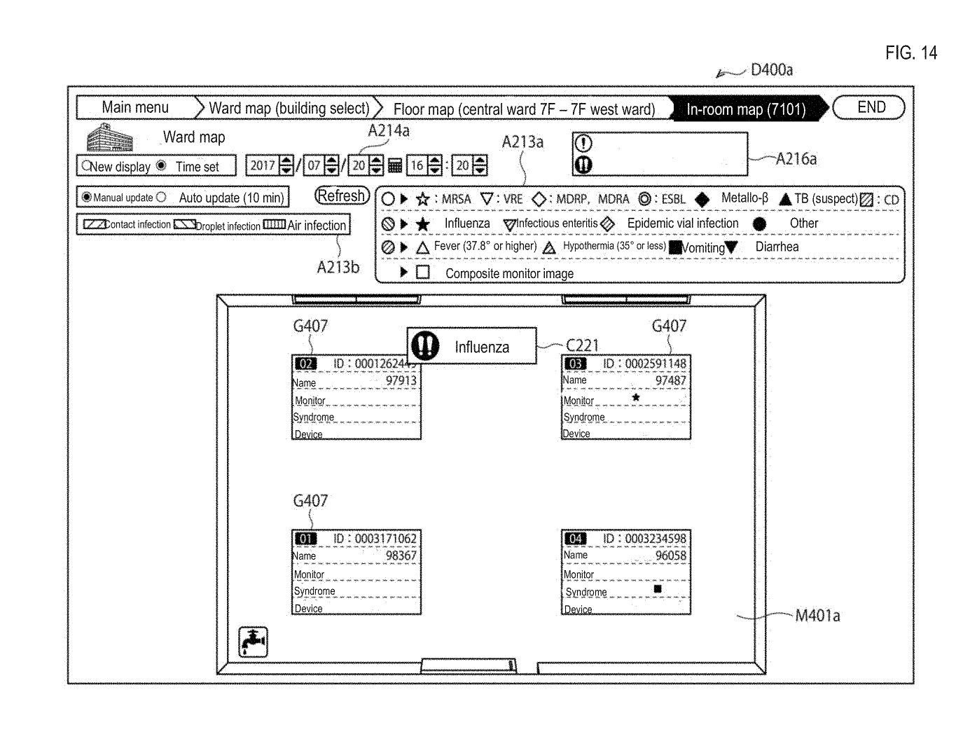

In the map display process 2, when the facility map icon C114 displayed on the main menu is selected in the client device 300, for example, the ward map screen D200a (FIG. 12) including the ward map M201a, the floor map screen D300a (FIG. 13) including the floor map M301a, and the patient room map screen D400a (FIG. 14) including the patient room map M401a are displayed on the display unit 32 of the client device 300.

The flow of the display process of the facility map is basically the same as the map display process 1, but the information related to the pathogen carrier, the type of the pathogen and the like, and the information on the presence or absence of the pathogen in the environment are acquired by the CPU 21a from the hospitalization information database DB7, the infectious disease test result database DB8, the environmental test result database DB9, and the attached device information database DB10.

### Ward Map

The configuration of the ward map in the map display process 2 is basically the same as the configuration described in map display process 1, but information on the pathogen carrier, the type of the pathogen and the like, and information on the presence or absence of the pathogen in the environment is added. Referring to FIG. 12, the ward map screen D200a is provided with a ward map area A202a in which the ward map M201a is displayed. A date and time designation area A214a for specifying the date and time and a caution alert legend area A216a are provided above the ward map area A202a. The date and time designation area A214a and the caution alert legend area A216a provided in the ward map area A202a are the same as the date and time designation area A214 and the caution alert legend area A216 provided in the ward map screen D200 shown in FIG. 8. A pathogen carrier number list display area A212a in which a list of the number of pathogen carriers in the whole hospital for each pathogen is displayed on the left side of the ward map area A202a. A legend area A213a in which a legend explaining the meaning of the symbols used in the ward map M201a is provided above the ward map area A202a. The legend area A213a is divided into groups such as bacterial infections, viral infections and the like according to the pathogens corresponding to each figure as a description of the figure indicating the pathogen carrier information G208a. Also, a legend area A213b for supplementary explanation of patterns of graphics used in the legend area A213a (for example, infection route such as contact infection, droplet infection, air infection) is provided on the left side of the legend area A213a. For example, in the legend region A213a, MRSA indicates that methicillin-resistant Staphylococcus aureus was detected. VRE indicates that vancomycin-resistant enterococci were detected. MDRP indicates multidrug-resistant Pseudomonas aeruginosa was detected. MDRA multi-drug resistant Acinetobacter was detected. ESBL indicates a substrate specific extended (β-lactamase) producing bacterium was detected. Metallo β indicates that metalloproteinase (MBL)-derived bacteria were detected. Tuberculosis (suspected) indicates the detection of Mycobacterium tuberculosis or atypical mycobacteria and suspected bacterial infection were detected. CD indicates that Clostridium difficile was detected. Influ indicates that influenza virus was detected. Infectious enterocolitis indicates that viral enteritis such as norovirus has been confirmed. Epidemic virus infection indicates that viral infection such as adenovirus and RS virus has been confirmed. Fever, hypothermia, vomiting, diarrhea indicate that the patient presenting the symptoms has been confirmed.

In the present embodiment, since a plurality of wards are provided in the medical facility, the ward switching tabs T203a and T204a are displayed in the ward map area A202a. The ward switching tabs T203a and T204a can be selected by a user's mouse click operation or the like, and the ward map M201a of the ward corresponding to the selected tab is displayed in the ward map area A202a. In the example shown in FIG. 12, each floor is displayed as in FIG. 8.

The ward map M201a may be displayed as a simplified figure of the entire ward. In the present embodiment, since the ward is configured by a plurality of floors, a ward map M201a showing the floors of each floor is displayed. A floor number display G215a is attached on the left side of the floor view G206a of each floor. In the floor view G206a, the floor information G207a is displayed for each floor. The floor information G207a includes the name G202a of each floor and the number G203a of patients who are hospitalized on the floor. In this way it is easy to comprehend which hospitalized patients are on which floor. When a plurality of floors are provided on the same floor, floor information G207a is displayed for each floor. In this way it is easy to comprehend which hospitalized patients are on which floor.

Similar to the map display process 1, caution alerts C221 and C222 on the floor also are displayed in the floor diagram G206a of each floor.

### Floor Map

Each floor information G207a can be selected by the user clicking the mouse. When one floor information G207a is selected, the facility map display process is called up and a floor map M301a of the selected floor is displayed.

Although the configuration of the floor map in the map display process 2 is basically the same as the configuration described in the display process 1 of the map, the information of the pathogen carrier, the type of the pathogen and information related to the presence/absence of pathogens in the environment has been added. In the example shown in FIG. 13, the floor map M301a is displayed on the floor map screen D300a. A legend area A213 in which a legend explaining the meaning of symbols is provided above the floor map M301a. A legend area A213b for supplementary explanation of the pattern of the figure used in the legend area A213a is provided on the left side of the legend area A213a. The legend areas A213a and 213b are the same as those in FIG. 12. Date designation area A214a, and caution alert legend area A216a are the same as those in FIG. 12. A legend area A215a indicating whether the patient is wearing a device also is provided under the legend area A213b.

The floor map M301 shown in FIG. 12 displays the structure of the entire floor as a simplified figure. In the floor map M301, the area information G307 is displayed for each area accommodating a patient. The area information G307 includes the area number of each area and the patient ID of the patients hospitalized in the area. When the patient is wearing a device such as a ventilator, a catheter, a blood transfusion route or the like, the patient ID is displayed in, for example, black and white inversion in the area information G307. When the patient is a pathogen carrier, a symbol G308a corresponding to the pathogen carried by the patient, which is shown in the legend area A213a, is displayed next to the patient's name.

In the floor map M301a, caution alerts C221 and C222 are displayed for each area corresponding to the area information G307a. The description of the caution alerts C221 and C222 is the same as the map display process 1.

### Patient Room Map

The user selects the area information G307a of the area desired to be displayed out of the area information G307a of the floor map M301a shown in FIG. 12 to call up the above map display process and display the patient room map M401a of the selected room can do.

Although the configuration of the patient room map in the map display process 2 is substantially the same as the configuration described in map display process 1, the information of the pathogen carrier, the type of the pathogen, and the presence or absence of the pathogen in the environment has been added. In the example shown in FIG. 13, the patient room map M401a is displayed on the patient room map screen D400a. A legend area A213 in which a legend explaining the meaning of the symbols is provided above the patient room map M301a. A legend area A213b for supplementary explanation of the pattern of the figure used in the legend area A213a is provided on the left side of the legend area A213a. The legend areas A213a and 213b are the same as those in FIG. 12. Date designation area A214a, and caution alert legend area A216a are the same as those in FIG. 12. A legend area A215a indicating whether the patient is wearing a device also is provided under the legend area A213b.

The patient room map M401a is displayed in a graphic form in which the entire structure in the patient room is simplified. In patient room map M401a, hospitalized patient information G407a such as the patient identification number (ID), patient name, infectious disease of the patient, symptoms (syndrome) presented by the patient and the like are displayed for each patient bed. When the patient wears a device, the type of the device worn by the patient is displayed in the “device” column of the hospitalized patient information G407a. A sensor 60 is arranged for each bed of a patient, and a caution alert C221 is displayed for each bed corresponding to the hospitalized patient information G407 in the patient room map M401 . The description of the caution alerts C221 and C222 is the same as the map display process 1.

### Caution Alert Display Process

When the facility map icon C114 is selected on the menu screen D100 shown in FIG. 7, the monitoring apparatus 200 transmits the warning alert display data 40 to the client device 300 in accordance with, for example, the following procedure, and the client device 300 causes the display unit 32 to display a warning alert. In the facility map displayed by the display unit 32, a warning alert is displayed based on the warning alert display data 40 created by the warning alert display process.

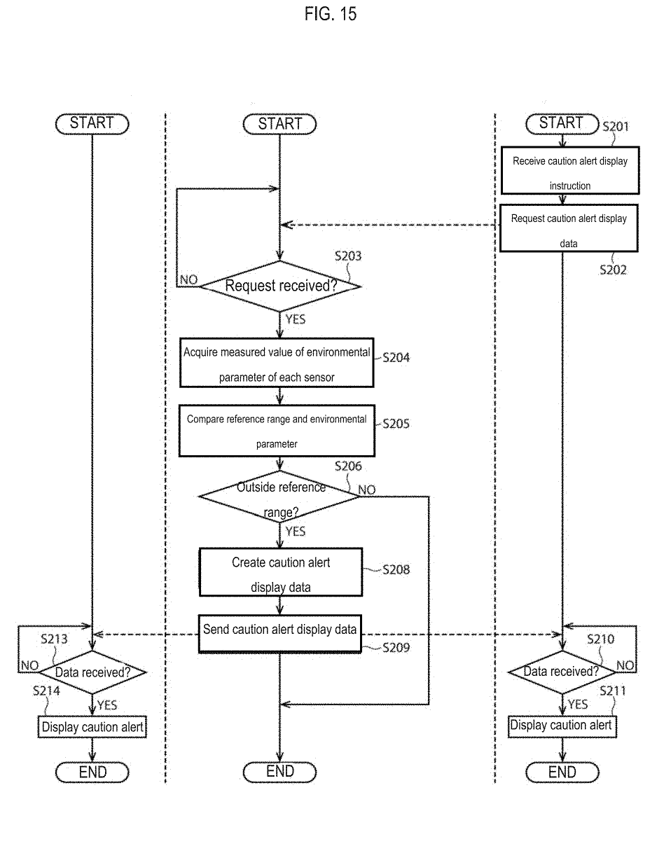

The processing flow will be described with reference to the flowchart shown in FIG. 15. In step S201, the facility map icon C114 is selected by input from the user's input unit 33, and the CPU 31a of the client device 300 accepts the caution alert display instruction.

In step S202, the CPU 31a of the client device 300 transmits a request instruction for requesting the caution alert display data 40 necessary for displaying the caution alert to the monitoring apparatus 200 via the communication interface 31g of the client device 300. The request instruction transmitted from the client device 300 is received by the monitoring apparatus 200 via the communication interface 21g.

In step S203, the CPU 21a of the monitoring apparatus 200 waits until it receives a request instruction.

When the monitoring apparatus 200 receives the request instruction, in step S204 the CPU 21a acquires the position information of each sensor 60 arranged in the facility from the sensor layout database DB5 and the positional information of each sensor 60 arranged in the facility, and measurement values of each environmental parameter 50 are acquired. In the determination of the environmental condition to be described later, when the measurement values of the environmental parameter 50 over time is used for determination of the environmental condition, the information of the measurement time is also acquired from the environment parameter relational database DB6. Here, the measurement values of each environmental parameter 50 may be temporarily stored in the RAM 21c.

In step S205, the CPU 21a compares the measurement value of each environmental parameter 50 with the reference range for each area in which each sensor 60 is provided, based on the acquired measurement values of each environmental parameter 50.

In step S206, the CPU 21a determines whether the measured value of each environmental parameter 50 is outside the reference range.

Although not shown, the CPU 21a also may include step S207 for determining in step S206 that the measured value of the environmental parameter 50 of an area is outside the reference range (YES) and the environmental condition is sub-nominal.

In step S208, the CPU 21a generates the caution alert display data 40 in consideration of the measured value of the environmental parameter 50 for the area in which the sensor 60 is provided and it was determined that the environmental condition is sub-nominal in step S207. Here, in step S208, the warning alert display data 40 also may be created (step S208) in consideration of the measured value of the environmental parameter 50 for the area in which the environmental condition was determined to be sub-nominal (YES) in step S207.

In step S209, the CPU 21a transmits the created caution alert display data 40 to the requesting client device 300 via the communication interface 21g. The caution alert display data 40 transmitted from the monitoring apparatus 200 is received by the client device 300 via the communication interface 31g. In step S209, the CPU 21a transmits the created caution alert display data 40 to the mobile terminal 400 via the communication interface 21g as necessary. The caution alert display data 40 transmitted from the monitoring apparatus 200 is received by the mobile terminal 400 via the communication interface 41g.

In step S210, the CPU 31a of the client device 300 waits until the caution alert display data 40 are received.

When the client device 300 receives the caution alert display data 40, in step S211 the CPU 31a causes the facility map to display a caution alert for the area in which the environmental condition is sub-nominal based on the received caution alert display data 40.

In step S213, the CPU 41a of the mobile terminal 400 waits until the caution alert display data 40 are received.

When the mobile terminal 400 receives the caution alert display data 40, in step S214 the CPU 41a displays a message notifying that the environmental condition is sub-nominal on the display unit 42 of the mobile terminal based on the received caution alert display data 40. Alternatively, the CPU 41a displays a caution alert for the area in which the environmental condition is sub-nominal together with the facility map.

If the measured values of all the environmental parameters 50 are within the reference range (NO) in step S206, the process is ended directly. Although not shown, a step S212 (not shown) for determining that the environmental state is nominal also may be included when the measured value of the environmental parameter 50 is within the reference range in step S206 (NO).

### Examples of Determination of Environmental Condition

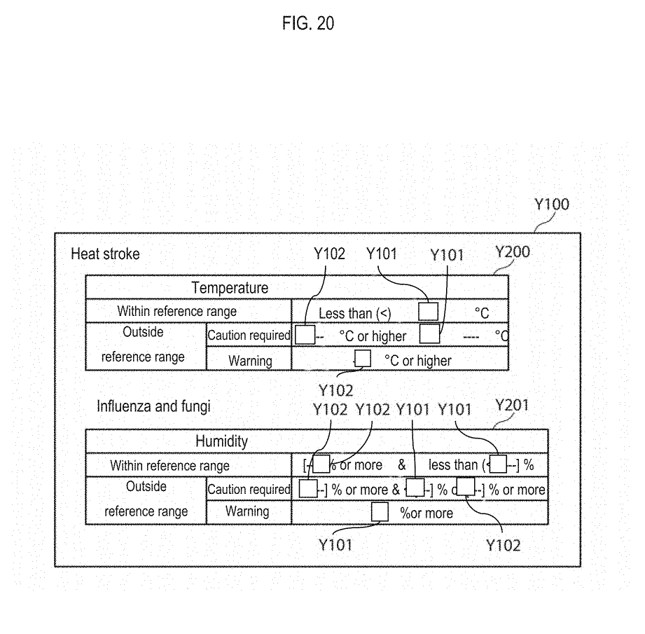

In step S205, there are various modes of the method by which the CPU 21a of the monitoring apparatus 200 determines the environmental condition. Five types of modes are exemplified below. The determination of the environmental condition is performed based on the reference range set according to the type of the disease that may occur due to deterioration of the environmental condition. The reference range may be set in accordance with the type of the disease, or different reference values may be set for each disease or for each disease. The user also may change the reference range for each disease or according to the facility or area. FIG. 20 shows a display example of the input screen when the user optionally sets the reference range. For example, an input area Y200 for inputting a reference range for monitoring heat stroke, and a reference range input area Y201 for inputting a reference range for monitoring influenza or fungi infection are provided on the input screen Y100 for optionally setting the reference ranges. An area Y101 for inputting the upper limit value of the reference range and an area Y102 for inputting the lower limit value of the reference range are provided in the reference range input areas Y200 and Y201. An optional numerical value is input to the areas Y101 and Y102 as the user operates the input unit 23.

The system 100 according to the embodiment of the invention monitors the environmental condition related to the occurrence of disease as the environmental condition measured at the sensor installation site. For example, various risks such as the susceptibility to viruses such as influenza virus, the ease of fungal and bacterial propagation, and the susceptibility to heat stroke are monitored. These three exemplified monitoring targets can be monitored, for example, based on the measurement value of the environmental parameter 50.

### 1) Determination Based on Temperature Information

In a first mode, the CPU 21a determines the environmental condition based on the temperature information, for example, in order to monitor susceptibility to the occurrence of heat stroke. Generally, heat stroke is more likely to occur as the temperature information in the compartment is higher. Therefore, in this embodiment, the CPU 21a determines that the environmental condition is sub-nominal for the area in which the temperature information is outside of the reference range.

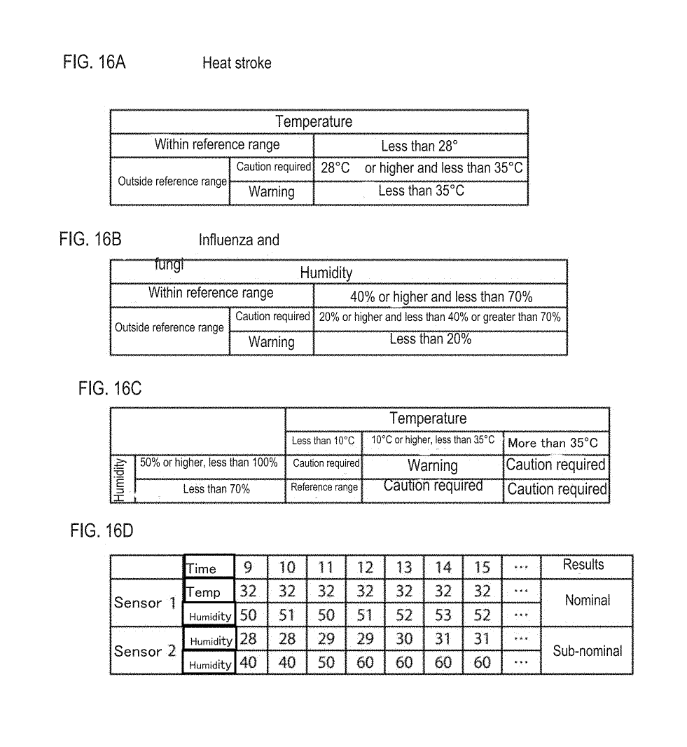

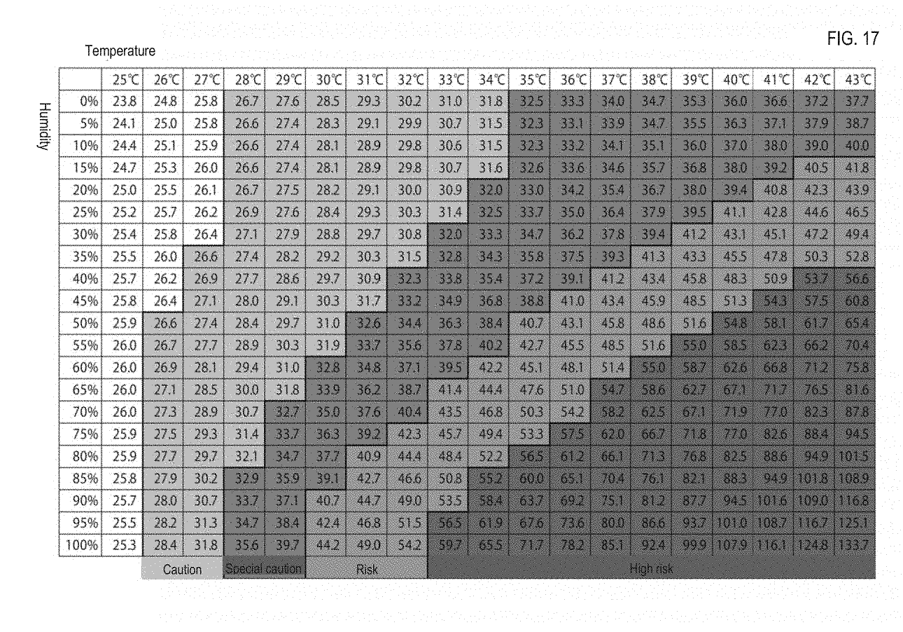

Specifically, if the measured value of the temperature acquired in step S204 of FIG. 15 is out of the reference range of the environmental parameter 50, the CPU 21a determines that the environmental condition is sub-nominal. For example, the reference range for determining that the environmental condition is nominal is set to be less than 28° C., and the table of the reference range data and the determination result shown in FIG. 16A is stored in the environment parameter relational database DB6. The CPU 21a compares the measured value of the temperature information output from each sensor 60 with the reference range stored in the environment parameter relational database DB6, and when the measured value of the temperature information is within the reference range, the environmental condition is determined to be nominal. When the measured value of the temperature information is outside of the reference range, the CPU 21a also determines that the environmental condition is sub-nominal. When it is determined that the environmental condition is sub-nominal, the CPU 21a can rate the measured value of the temperature information according to the degree of danger of the environmental condition in which heat stroke occurs. For example, as shown in FIG. 16A, the range of the temperatures at which the danger degree of the environmental condition is judged to be “warning level” is set to 28° C. or more and less than 35° C.; the CPU 21a can determine that the environmental condition is “caution required” or “warning” in accordance with the degree of danger of the environmental condition by setting the range of the temperature at which the risk level of the environmental condition is determined to be “warning” to 35° C. or higher and storing it in the environment parameter relational database DB6.

### 2) Determination Based on Humidity Information

In a second mode, the CPU 21a determines the display condition of the caution alert based on the humidity information in order to monitor the susceptibility to viruses such as influenza virus and the ease of fungus propagation. It is known that fungi easily propagate when the humidity is about 70% or more, and influenza susceptibility increases when the humidity becomes less than about 40%. Therefore, in this embodiment, the CPU 21a determines that the environmental condition is sub-nominal for the area in which the humidity information is outside of the reference range.

Specifically, when the measured value of the humidity information acquired in step S204 is outside the reference range, the CPU 21a determines that the measured value satisfies the condition related to the occurrence of the acquired disease. For example, the reference range for determining that the environmental condition is nominal is set to 40% or more and less than 70%, and the reference range data and the determination result are stored in the environment parameter relational database DB6 with the reference range data and the determination result table shown in FIG. 16B. The CPU 21a compares the measured value of the humidity information output from each sensor 60 with the reference range stored in the environment parameter relational database DB6, and the environmental condition is determined to be nominal when the measured value of the humidity information is within the reference range. When the measured value of the humidity information is outside of the reference range, the CPU 21a also can determine that the environmental condition is sub-nominal. When it is determined that the environmental condition is sub-nominal, the CPU 21a can rate the measured value of the humidity information according to the risk of susceptibility to influenza virus. As shown in FIG. 16B, for example, by setting the range of humidity for determining that the degree of risk of the environmental condition “caution required” to 20% or more and less than 40%, or 70% or more, and, for example, setting the range of humidity for determining the degree of risk of the environmental condition “warning” to be less than 20% and storing these settings in the environmental parameter relational database DB6, the CPU 21a can determine whether the environmental condition is “caution required”, or “warning”.

### 3) Combining Temperature Information and Humidity Information

In the third mode, the CPU 21a determines the display conditions of the caution alert based on the temperature information and the humidity information, for example, in order to monitor the ease of propagation of fungi and bacteria. It is known that fungal bacteria propagate in a state of, for example, a temperature of 20° C. to 30° C. and humidity of 70% or more. Therefore, in this mode, the CPU 21a determines that the environmental condition is sub-nominal for the area in which both the temperature information and the humidity information are outside of the reference ranges.

Specifically, when the measured value of each environmental parameter 50 is out of the reference value range with respect to the measured values of the temperature information and the humidity information acquired in step S204, the CPU 21a determines that the environmental condition is sub-nominal. For example, a reference range for determining that the environmental condition is nominal is set to be a temperature of less than 10° C. and a humidity of less than 70% and, for example, the table of reference range data and determination results shown in FIG. 16C is stored in the environment parameter relational database DB6. The CPU 21a compares the measured values of the temperature information and the humidity information output from the respective sensors 60 with the reference ranges stored in the environment parameter relational database DB6, and determines that the environmental condition is nominal when the measured values of the temperature and the humidity are within the reference ranges. When the measured values of the temperature information and the humidity information are outside of the reference ranges, the CPU 21a can determine that the environmental condition is sub-nominal. When it is determined that the condition related to the occurrence of disease is satisfied, the CPU 21a can rank the environmental condition according to the risk of fungus or bacteria propagation based on the measured values of the temperature information and the humidity information. For example, as shown in FIG. 16C, the range determining the degree of risk of the environmental condition is “caution required” is set to be within a range of 10° C. or more and less than 35° C., and the range of humidity is 70% or more and less than 100% (more preferably, the temperature is in the range of 20° C. or more and less than 30° C., and the humidity is in the range of 80% or more and less than 100%). For example, the range of the humidity for determining that the risk level of the environmental condition is “warning” is set to such that the temperature is within a range of 10° C. to 35° C., and the humidity is set in a range of 70% or more and less than 100% (more preferably, the temperature is in the range of 20° C. or more and less than 30° C. or less, and the humidity is in the range of 80% or more and less than 100%). By storing in the environment parameter relational database DB6 in this manner, the CPU 21a can determine that the environmental condition is “caution required” or “warning” according to the degree of risk presented by the environmental condition.

For example, fungi include dry fungus and wet fungus, and when the temperature is within the range of about 10° C. to about 40° C. and the humidity exceeds about 90%, both wet and dry fungi propagate regardless of the type of fungi. Therefore, with respect to fungi, the range indicated as “warning” as described above may be in the range of 10° C. or higher and less than 40° C., and the humidity may be in the range of 90% and higher and less than 100%.

### 4) Determination Based on Index

In the fourth mode, it is determined whether the environmental condition is nominal or not based on an index obtained from the measured value of each environmental parameter 50. The index is a value for rating an environmental condition derived by combining a plurality (two or more)measurement values of environmental parameters 50. An example of determining the risk of occurrence of heat stroke by using the sensible temperature obtained based on the temperature information and the humidity information will be described as an example of an index.

The risk of heat stroke is related not only to temperature but also to humidity, and the heat index (Heat Index), which is an index adopted by the National Weather Service (NWS) in the United States, is a sensible temperature obtained from temperature and humidity. That is, the sensible temperature is an index in the fourth mode. For example, a heat index table exemplified in FIG. 17 is stored in the environment parameter relational database DB6. The CPU 21a obtains the sensible temperature based on the temperature information and the humidity information output from each sensor according to the table of the heat index. In the heat index table exemplified in FIG. 17, the reference range is 26.4° C. or less. Therefore, the CPU 21a compares (checks) the obtained sensible temperature with the table of the heat index, and when the sensible temperature is within the reference value range, and determines the environmental condition is nominal. When the sensible temperature is outside of the reference range, the CPU 21a can determine that the environmental condition is sub-nominal. When the environmental condition is determined sub-nominal, for example, the sensible temperature can be rated according to the risk of occurrence of heat stroke. When the environmental condition is determined sub-nominal, the sensible temperature can be rated according to the degree of risk of the environmental condition where heat stroke occurs. For example, a range in which the degree of risk of the environmental condition is determined as “caution”, the range determined as “special caution”, the range judged as “risk”, and the range determined as “high risk” are set in the heat index shown in FIG. 17.

Therefore, by storing the table of FIG. 17 in the environment parameter relational database DB6, the CPU 21a can determine the environmental condition to be “caution”, “special caution”, “risk”, or “high risk”d.

### 5) Determination Based on Measured Values of Temperature Information and Humidity Information Over Time

For example, it is said that influenza virus infection is less likely to occur when conditions are maintained at a temperature of 32° C. or more and a humidity of 50% or more for several hours within a day. Therefore, it is possible to prevent spread of influenza virus infection by keeping the interior of each area temporarily in such under these conditions. In this embodiment, whether the desirable environmental condition is maintained for a predetermined period is considered in the determination of the environmental condition. Specifically, whether the environmental condition of each section is nominal or sub-nominal is determined based on the measured values of the environmental parameter 50 stored over time. Here, when a person is staying in an area, it is possible to move the person outside the area as necessary.

In step S204, the CPU 21a acquires the measurement value of the environmental parameter 50 output from each sensor 60 over time and stores it in the storage unit 21d or the RAM 21c as shown in FIG. 16D, for example. In step S206, the CPU 21a determines whether there is a time outside the reference range (for example, a temperature of 32° C. or more and humidity of 50% or more) in the measured values of the predetermined period stored in the storage unit 21d or the RAM 21c, it can be determined that the condition related to the occurrence of the disease is not satisfied when there is no time that the measured value is outside the reference range. Here, the predetermined period for making the determination depends on the disease to be monitored. In step S206, when there is time that is outside the reference range within a predetermined period, the CPU 21a can determine that the environmental condition is sub-nominal. In step S206, the CPU 21a determines an index obtained from the measured values of the environmental parameter 50, determines whether the index of the environmental parameter 50 is outside of the reference range within the predetermined period, and determines whether the environmental condition is nominal or sub-nominal.

When it is determined that the environmental condition is sub-nominal, the range for determining the degree of risk of the environmental conditions of “caution required” and “warning” can be appropriately determined according to the degree to which the measured value or index of the environmental parameter 50 deviates from the reference range or how many times it has deviated.

When the environmental condition is determined to be “warning” in the first to third and fifth modes, in step S208 the CPU 21a creates a caution alert C221 meaning “warning” as the caution alert display data 40. When the condition of the indoor environment is determined to be “caution required”, in step S208 the CPU 21a creates a caution alert C222 meaning “caution required” as the caution alert display data 40. In the fourth mode, the caution alert display data 40 corresponding to the range of “caution”, “special caution”, “risk”, and “high risk” are created.

In step S212, for an area in which the environmental condition is determined to be “nominal”, a caution alert is not displayed in the corresponding area in the facility map.

Five types of modes for generating the caution alert display data have been described above. Note that any one of these conditions may be used in order to display a caution alert, or a plurality may be selected and used from these conditions.

The operation of the system also includes, as an optional step, a further step for restoring the environmental condition in the area to a nominal state, for example, when it is determined in step S206 that the environmental condition in the area is sub-nominal. Specifically, this step causes the CPU 21a of the monitoring apparatus 200 to execute functions to generate a command for controlling the air conditioning system or the lighting system or the like in the facility or the area, and transmit the command to the air conditioning system or the lighting system or the like. Alternatively, a further step for restoring the environmental condition in the area to a nominal state is cause the CPU 31a of the client device 300 to execute functions generate a command to control the air conditioning system or the lighting system or the like in the facility or the area, and to transmit the command to the air conditioning system or lighting system.

### Computer Program

One embodiment of the invention relates to a computer program for causing a computer to function as a monitoring apparatus 200.

In the computer program according to the embodiment, at least in response to a request from the client device 300, the control unit 21a of the monitoring apparatus 200 causes the processes of steps S203 to S209 shown in FIG. 15 to be executed, and generates caution alert display data at least in response to a request from the client device 300. The computer program according to this embodiment also may cause the control unit 21a of the monitoring apparatus 200 to execute the processes of steps S103 to S106 illustrated in FIG. 11 to generate map display data.

The program also may include, as an optional step, a further step for restoring the environmental condition in an area to a nominal state when it is determined in step S206 that the environmental condition in the area is sub-nominal. Specifically, this step causes the CPU 21a of the monitoring apparatus 200 to execute functions to generate a command for controlling the air conditioning system or the lighting system or the like in the facility or the area, and transmit the command to the air conditioning system or the lighting system or the like.

The computer program according to the embodiment may be stored in a storage medium such as a hard disk, a semiconductor memory element such as a flash memory, an optical disk or the like. The storage format of the program in the storage medium is not limited insofar as the information acquisition device can read the program. Storage in the storage medium is preferably nonvolatile.

### In-Facility Monitoring Method

This embodiment relates to an in-facility monitoring method. In the method, includes a step of generating display data in order to identifiably display the environmental conditions in each area in the area layout diagram based on the measured values of the environmental parameter acquired from the sensors provided for each of the plurality of areas arranged in the facility. The step of identifiably displaying the environmental conditions also may be included in the area layout diagram.

The embodiment also may include a step of acquiring a measured value of an environmental parameter for comprehending the environment in an area for each of a plurality of areas arranged in the facility before generating the display data. An area in which the measured value of the environmental parameter is outside the reference range may be identifiably displayed in the area layout diagram.

In the embodiment, the measured value of the environmental parameter can be obtained from the above-described sensor. The embodiment also may include a step of determining whether the measured value of the environmental parameter is outside the reference range for each area after the acquisition step and before the display step.

In the present embodiment, it is possible to determine whether measured value of the environmental parameter is outside the reference range according to the determination example of the environmental condition.

The description of the invention in the above Summary of the Invention, System Configuration, System Operation and the like are all hereby incorporated by reference.

### Other Embodiments

Although the invention has been described with reference to specific embodiments, the present invention is not limited to the above-described embodiments.

Although the in-facility monitoring system 100 is configured as a system in which the monitoring apparatus 200 and the client device are connected to each other via a communication network in the above embodiment, the configuration of the in-facility monitoring system 100 is not limited thereto. The in-facility monitoring system 100 also may be configured such that the monitoring device 200 and the client device 300 are integratedly configured.

Although the monitoring apparatus 200 is realized as a single apparatus in the above embodiments, the monitoring apparatus 200 is not necessarily an integrated apparatus. The monitoring apparatus 200 may be realized as a distributed type device in which the CPU 21a, the RAM 21c, the storage unit 21d and the like are arranged at different locations, and these are connected via a communication network. Similarly, for the client device 300, the client device 300 is not necessarily an integrated device. The client device 300 also may be realized as a distributed type device in which the CPU 31a, the RAM 31c, the storage unit 31d, the display unit 32, the input unit 33 and the like are arranged at different locations, and these are connected via a communication network. That is, the in-facility monitoring system 100 may be realized as a distributed system including a control unit of the monitoring apparatus 200 and a display unit 32 of the client device 300.

Although the monitoring apparatus 200 executes processing by a single CPU 21a in the above embodiments, the present invention is not limited to this configuration. The monitoring apparatus 200 also may distribute and execute processing by a plurality of CPUs. Similarly, regarding the client device 300, the client device 300 may distribute and execute processing by a plurality of CPUs.

Although the client device 300 is realized by a stationary computer in the above embodiments, the present invention is not limited thereto. The client device 300 also may be realized as a portable computer such as a smart-phone, a tablet device, a laptop computer or the like.

Although one client device 300 is provided in the in-facility monitoring system 100 in the above embodiments, it is not limited thereto. For example, a plurality of client devices 300 may be arranged for each floor in a hospital (for example, for each nurse station). Similarly, regarding the wireless device 70, a plurality of wireless devices 70 may be arranged, for example, for each floor in a hospital (for example, for each room).

Although the monitoring apparatus 200 determines the environmental condition based on the measured value of the acquired environmental parameter or the index obtained from the measured values in step S205 of the process of displaying the caution alert in the above embodiments, the embodiments of the invention is not limited thereto. For example, the actual measured value of the sensor 60 also may be corrected according to the temperature and humidity outside the facility, the season at the time of measurement, the weather conditions, the time and the like, and compare it with the reference range based on the measured value of the amended parameter after the correction. For example, weather situation data can be fetched from a data server of the Japan Meteorological Agency via the communication network 80 and used for correction. In addition to correction of actual measured values, the reference range also may be corrected according to temperature and humidity outside the facility, seasons at the time of measurement, weather conditions, time and the like. As to when to perform such a correction process, the date and time of processing may be stored in the storage unit 21d of the monitoring apparatus 200 in advance. The date and time, the period, and the time interval for measuring the environmental parameter can be determined in consideration of the time when influenza is prevalent, the time when heat stroke occurs, and the time when fungal propagation becomes active. For example, in Japan, between March and May and between September and October scheduling is performed at a 48-hour cycle, from November to February scheduling is performed at a 24-hour cycle, and from June to August scheduling is performed at a 48-hour cycle or longer.

Although the caution call levels indicated by the caution alerts C221 and C222 are indicated by exclamation points surrounded by circular frames in the above embodiments, the display mode of the caution call level is not limited to this. Instead of an exclamation point, the caution call level may be displayed using other marks such as an asterisk. Furthermore, not only the mark but also the caution call level may be displayed using a color, icon, character, or numerical value. The caution call level also may be displayed as a message, particularly when displaying a caution alert on the display unit 42 of the portable terminal 400. Information for identifying an area with a sub-nominal environmental condition and/or an item for an environmental parameter outside the reference range also may be displayed in the message.

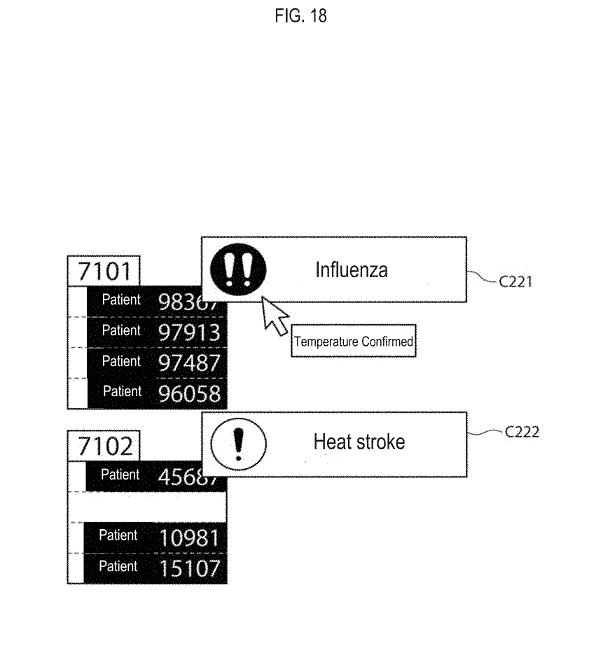

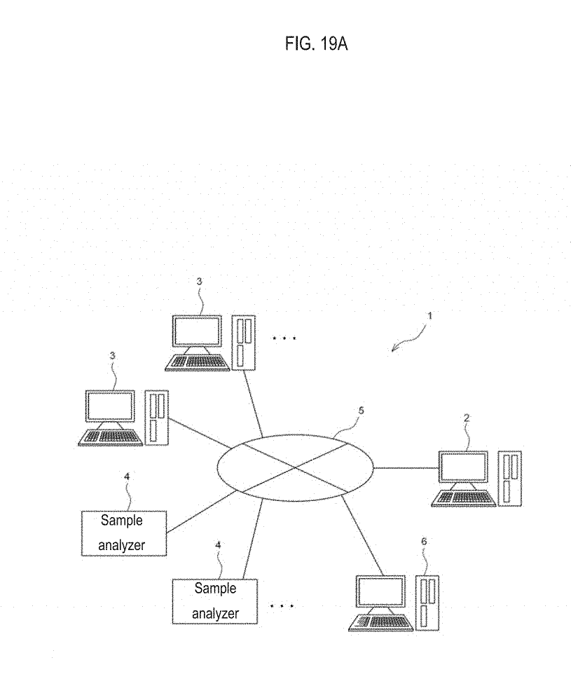

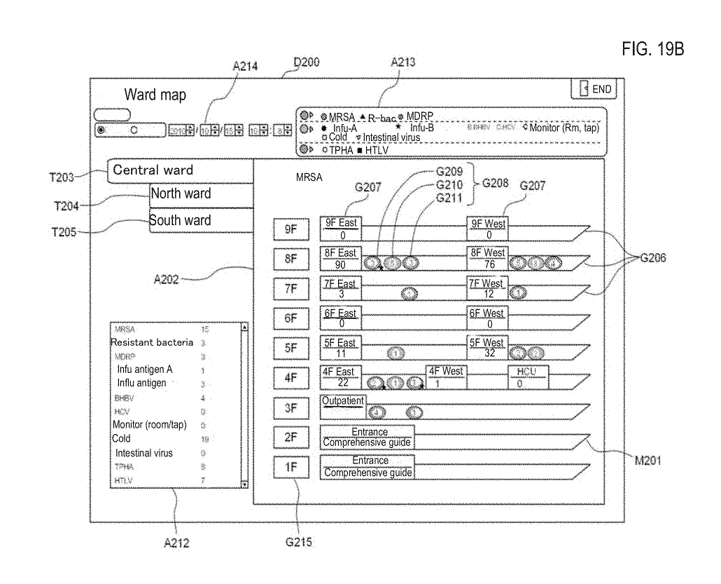

Although the caution alert is displayed without distinguishing between susceptibility to viruses such as influenza viruses to be monitored, susceptibility to propagation of fungi or bacteria, and susceptibility to the occurrence of heat stroke in the above embodiment, the invention is not limited to this mode. For example, a plurality of monitored targets may be distinguished and displayed by constructing a caution alert with an exclamation mark and a combination of colors. As shown in FIG. 18, when a mouse is pointed at a caution alert, it also is possible to display which item of the environmental parameter is the reference range.

Although the CPU 21a of the monitoring apparatus 200 creates the caution alert display data 40 for each sensor 60 in step S208 in the above embodiments, the invention is not limited thereto. The monitoring apparatus 200 also may transmit the determination result of the environmental condition to the client device 300 instead of transmitting the caution alert display data 40. In this case, the client device 300 records the caution alert display data 40 in advance in the storage section 31d, appropriately selects the caution alert display data 40 according to the determination result of the received environmental condition, and displays the data on the facility map.

Although the caution alerts C221 and C222 are displayed on the facility map with icons n the above embodiments, the mode of displaying the caution alerts in the facility map is not limited. For example, instead of displaying the icons, the caution alert also may be displayed by changing the display mode of the floor information G207, the area information G307, and the hospitalized patient information G407 that must display the caution alert. For example, various modes such as a mode of display using shading of colors, a mode of display using a specific color, a specific mark, character, sound, vibration, display blinking, and dynamic icon display changes and the like may be used as a mode of displaying the caution alert.

Although the state of the indoor environment is indicated by displaying the caution alerts C221 and C222 in the facility map in the above embodiments, the present invention is not limited to this mode.

Although the sensor 60 measures environmental parameters at predetermined time intervals, and the monitoring apparatus 200 automatically updates the display content of the facility map and the display content of the caution alerts in the above embodiment, the predetermined time interval Is not particularly limited. For example, the time interval may be set to be shorter for an area in which a standard time interval is set to 10 minutes and an elderly patient or an infant patient is hospitalized. The age of the patient can be acquired from the existing electronic medical record system in the facility by connecting the monitoring apparatus 200 to the electronic medical record system.

### EXPLANATION OF THE REFERENCE NUMERALS

32 Display unit
40 Display data
60 Sensor
21a Control unit
21d Storage unit
50 Environmental parameter
100 In-facility monitoring system
200 In-facility monitoring apparatus
300 Client device
Mobile Terminal

What is claimed is:

1. An in-facility monitoring system comprising:
a display unit for displaying an area layout diagram showing the positions of a plurality of areas in the facility;
a sensor which is provided in each of the areas and measures environmental parameters related to the risk of occurrence of disease; and
a control unit for generating display data for identifiably displaying an environmental state representing the risk of disease occurrence in each area on the basis of the measurement value of the environmental parameters output from the sensor in the area layout diagram.

2. The system according to claim 1, wherein
the storage unit stores a reference range of environmental parameters; and
the control unit determines that the environmental condition is sub-nominal for an area in which the measured value of the environmental parameter falls outside the reference range.

3. The system according to claim 1, wherein
the storage unit stores a reference range of environmental parameters; and
the control unit obtains an index representing an environmental condition indicating the risk of occurrence of a disease in each area from the measured value of the environmental parameter, and determines that the environmental condition is sub-nominal for the area in which the index exceeds the reference range.

4. The system according to claim 2, wherein
the storage unit stores a reference range corresponding to the types of the disease.

5. The system according to claim 1, wherein
the environmental parameter is at least one selected from a group comprising temperature information, humidity information and ventilation information.

6. The system according to claim 1, wherein
the illness is an infection or heat stroke.

7. The system according to claim 1, wherein
the infection is a droplet infection.

8. The system according to claim 7, wherein
the droplet infection is caused by at least one selected from a group including viruses, fungi, and bacteria.

9. The system according to claim 8, wherein
the virus is at least one member selected from a group including influenza virus, RS virus, adenovirus, rhinovirus, coronavirus, and parainfluenza virus;
the fungus is at least one member selected from the group including Candida, Actinomyces, Geotrichum, Aspergillus, Cryptococcus, and Nocardia; and
the bacterium is at least one selected from a group including Mycoplasma, Legionella, Haemophilus, Pneumococcus, and Bordetella pertussis.

10. The system according to claim 2, wherein
the sensor measures the environmental parameter over time;
the control unit records the measured value of the environmental parameter measured by the sensor over time in the storage unit, and determines whether the environmental condition of each area is nominal or not based on the measured value of the environmental parameter over time.

11. The system according to claim 1, wherein
the control unit generates caution alert data for identifiably displaying a caution call level according to the environmental condition of each area of the in-facility area diagram.

12. The system according to claim 11, wherein
the caution call level is identifiably displayed by a mark, color, icon, character, or numerical value.

13. The system according to claim 1, wherein
the control unit notifies the preset mobile terminal of the environmental state of the corresponding area when there is an area with a sub-nominal environmental condition.

14. The system according to claim 1, wherein
the control unit causes the display unit to display the environmental parameter of a corresponding area when there is an area having a sub-nominal environmental condition.

15. The system according to claim 1, wherein
the facility can accommodate a plurality of people.

16. The system according to claim 1, wherein
the facility is at least one selected from a group including a medical facility, an educational facility, and a welfare facility.

17. The system according to claim 1, wherein facility is a medical facility; and
the area is a room.

18. An in-facility monitoring apparatus comprising:
a storage unit and a control unit, wherein
the apparatus is connected to a client device having a display unit;
the storage unit stores an area layout diagram showing positions of a plurality of areas in a facility; and
the control unit acquires environment parameters related to the risk of occurrence of diseases in each of the areas output from the sensors provided for each of the areas, generates display data identifiably display the environmental conditions representing the risk of occurrence of disease in each area on the area layout diagram based on the acquired environmental parameters, and transmits the created display data to the client apparatus.

19. An in-facility monitoring method comprising:
a step of generating display data for identifiably displaying environmental conditions indicating the risk of occurrence of disease in each area in an area layout diagram showing the position of each area in the facility based on the measured values of the environmental parameters related to the risk of occurrence of disease in the area acquired from the sensors provided for each of the plurality of areas in the facility.

20. The method according to claim 19, further comprising:
a step of identifiably displaying the environmental conditions on the area layout diagram after the display data generating step.
